(19) United States
(12) Patent Application Publication
(10) Pub. No.: US 2010/0062438 A1
(43) Pub. Date: Mar. 11, 2010

(54) NATURAL SELECTION AND CELLULAR IMMORTALITY

(75) Inventor: Antoine DANCHIN, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: INSTITUT PASTEUR, Paris Cedex (FR)

(21) Appl. No.: 12/508,753

(22) Filed: Jul. 24, 2009

Related U.S. Application Data
(60) Provisional application No. 61/083,282, filed on Jul. 24, 2008.
(60) Provisional application No. 61/083,266, filed on Jul. 24, 2008.

(51) Int. Cl. C12Q 1/68

(52) U.S. Cl. 435/6

(57) ABSTRACT

The invention provides new insights into the manner in which cells evolve and age thereby providing methods for assessing and studying those processes.

### CROSS-REFERENCE TO RELATED APPLICATIONS

The present applications claims the benefit of U.S. provisional applications 61/083,266 and 61,083,282 both filed on Jul. 24, 2008 and both of which are incorporated herein by reference.

### BACKGROUND OF THE INVENTION

1. Field of the Invention

The present invention provides new insights into the manner in which cells evolve and age thereby providing methods for assessing and studying those processes.

2. Description of the Related Art

All living organisms—bacteria included—age and eventually die. Our usual anthropocentric view of Nature (Koyré 1973) has led us to study ageing essentially in multicellular organisms, and more particularly in organisms where (some) individuals carry the germ line and propagate the species, while they become old and pass away. I will try here to see this process as much more general—after all at least 50% of the Earth's protoplasm is made of microbes—and investigate its relationship with the general constraint that operates on all living organisms, and that we know as Natural Selection. To this aim I will begin my investigation at the lowest level relevant for the study of life, the molecular level.

Whereas there is a tendency to see senescence as a fairly slow process, chemical analysis of proteins shows that many proteins tend to age extremely rapidly, even in the absence of exogeneous stresses such as Reactive Oxygen Species (ROS) (Rattan 2008). A major cause of protein ageing is spontaneous isomerisation of aspartate and asparagine, leading to the presence of isoaspartate residues (with concomitant deamidation in the case of the latter aminoacid) in the alpha carbon chain, distorting the protein backbone (Shimizu et al 2005; Galletti et al 2007). In many organisms, including humans, this process is counteracted by an energy-costly repair system involving S-adenosylmethionine-mediated methylation of isoaspartate and reversion to aspartate (Clarke 2003). The role of this repair system is so central that its deficiency leads in mammals to lethal diseases such as fatal progressive epilepsy (Yamamoto et al 1998). Despite the extreme importance of this process, not much study has been devoted to its overall extent and influence on other general biological processes. In bacteria such as Escherichia coli, the isomerisation of aspartate is extremely rapid in some proteins. In ribosomal protein S11, for example, aspartate is converted into isoaspartate within minutes, suggesting (but by no means proving) that this isomerisation might belong to the normal function of the protein (David et al 1999). In some proteins this cyclisation results in a self-cleavage step essential for the function of the protein (Zarivach et al 2008). During stationary phase, isoaspartate residues steadily accumulate.

This is consistent with recent experiments which show that contrary to a universally spread belief, bacteria, as all living organisms, would age even when other complex modifications such as ROS-mediated modifications or protein glycation are not taken into account ((Nystrom 2003; 2007; Lindner et al 2008), see also (Holliday 2006; Partridge 2007) for a general discussion of ageing in single cell and multicellular organisms). A further indication of the importance of this molecular process was found in organisms living in extreme environments. This isomerisation process being temperature-dependent, it was interesting to explore its importance in cold-living bacteria. As a matter of fact, asparagine residues are accumulating significantly in psychrophiles such as Pseudoalteromonas haloplanktis (which can grow fast at 0° C.) (Medigue et al 2005) and Psychromonas ingrahamii (which can grow at temperatures as low as −12° C.) (Riley et al 2008), showing both that this amino acid must have some physico-chemical property important for protein structure (such as formation of asparagine ladders (Jenkins and Pickersgill 2001)) that is counterbalanced by its tendency to age.

These observations imply that all organisms, including bacteria that reproduce by morphologically symmetric division, age (Stewart et al 2005), and age fast. A consequence is that extant organisms are always composed with a significant proportion of aged structures, with the remarkable implication, rarely emphasized, that in the chain of descent, new organisms are always born from aged ones, sometimes as patchworks of aged and young structures. This may happen in various ways, sometimes with daughter cells somewhat similar to each other, especially when division looks superficially symmetrical (which it is not, in reality (Rocha et al 2003; Stewart et al 2005; Fuentealba et al 2008; Lindner et al 2008)), sometimes with a mother cell carrying most, if not all of the aged structures (Aguilaniu et al 2003). This inevitable process opens the apparent paradox that aged biochemical structures (ribosomal proteins age, as just mentioned, and this may have enormous consequences as ribosomes are the central factories making proteins (Orgel 1963; Rattan 1996)) direct synthesis of young ones.

In a seminal reflection about the constraints operating at the origin of life, where chemical processes could not be as accurate as they are in extant organisms, Freeman Dyson showed that while replication inevitably leads to the accumulation of errors and the error-catastrophe (Orgel 1963), reproduction is not doomed to lead to progressively less efficient processes, but rather, can progressively improve their efficiency (Dyson 1985). At the other extreme of chemical process that resulted in life, a similar distinction has also been considered in general reflections about the existence or role of ageing in life history strategies of a most recent domain of life, that of multicellular organisms (Stearns 1992). Although highly relevant to the conjectures detailed here, and in order to make the question simpler, I shall not explore the latter in depth, but, rather, explore the fate of single cell organisms (see however some comments at the end of this essay).

Following a trend that has fallen in disuse (see the role of Schrödinger's book at the origin of molecular biology (Schrödinger 1945)), I shall also proceed in a somewhat unusual way at the present stage of biological studies, in placing explicitly life processes within the realm of Physics. This follows the most recent developments of Physics, that include information as a fifth component of Nature, which has to be superimposed on the four traditional ones, matter, energy, space and time (Steane 1998). Considering the model of the cell as a computer making computers, this novel trend fits remarkably well the separation between the genetic program, which replicates, and the machine that reads the program, which reproduces (Danchin 2009). As a matter of fact, while the genome of a daughter cell tends to be a replica of its parent, the daughter cells are not a replica of their mother-cell (Rocha et al 2003; Lindner et al 2008), even in eukaryotic cells after mitosis (Fuentealba et al 2008).

There are many definitions of ageing, mostly associated to the type of organism considered and heavily dependent on the entities considered within the cell. More often than not, it has been assumed that bacteria do not really age, at least when they are growing exponentially. While this was long suspected (Lam et al 2006), it is now firmly established that division is not symmetrical and that bacteria do age, even in these circumstances ((Stewart et al 2005) and see discussion in (Stewart and Taddei 2005)). The question of ageing has however often been restricted to the case of multicellular organisms where there is an obvious distinction to be made between the organism and its germ-line. The “disposable soma theory” proposes that what we witness as ageing is essentially a side effect of longevity: reproduction of young organisms permits the organism to escape death of the soma, which rapidly remains a disposable leftover that may, under favorable circumstances display significant longevity (Kirkwood and Holliday 1979). In this context ageing is considered by many as somewhat irrelevant to the problem of perpetuation of the species, but, rather as essentially a problem faced by the individual organism, whether it reproduces or not. This reflection is particularly adapted to the case of individual organisms that have a role in the group but are not reproducing, such as is the case in social insects. It is of course central to the reflection of individual human beings, who are doomed to face the burden of this inevitable process.

Rather than discuss ageing in this light, I accept the recent experimental data that demonstrate that bacteria age and take a slightly different stance, considering the way components in the cell age, with single-cell bacteria as reference organisms to see whether and how this relates to the way genomes are organized. I explore the reproduction/replication dialogue, basing my reflection on a deep analysis of the structure of bacterial genomes and see how this is related to the process of ageing at the molecular level.

The core of the model constructed by Dyson rests on the demonstration that reproduction predates replication and can improve over time, but the model does not propose explicit implementation of the concrete process (Dyson 1985). It shows mathematically how prebiotic metabolic systems could progressively become more and more accurate, before they could discover replication. Interestingly, this approach is conceptually very similar to the viewpoint taken in the disposable soma theory, with a completely different background. The argument I wish to develop here is to show how the creation of a link between these views and information theories can lead to very concrete—experimentally testable—hypotheses.

Making young entities from aged ones implies creating (or recovering) some information. Despite the sociological separation between the domains of Physics and Biology the latter rests on all the constraints imposed by the former. It is therefore natural to place Biology in the context of recent developments in Physics to explore whether this would help getting deep insights into deep questions of Biology, while proposing experiments that would lead us to better understanding. Before getting into the heart of the matter, we thus need to revisit some of the concepts of Information Theory. This is particularly fit when considering genomes from the algorithmic point of view that underlies the function of the genetic program.

### SUMMARY OF THE INVENTION

Genomes replicate while the host cells reproduce. I explore the reproduction/replication dialogue, based on a deep analysis of bacterial genomes, in relation to ageing. Making young structures from aged ones implies creating information. I revisit Information Theory, showing that the laws of physics permit de novo creation of information, provided an energy-dependent process preserving functional entities makes room for entities accumulating information. I identify explicit functions involved in the process and characterize some of their genes. I suggest that the energy source necessary to establish reproduction while replication is temporarily stopped could be the ubiquitous polyphosphates. Finally, I show that rather than maintain and repair the original individual, organisms tend to metamorphose into young ones, sometimes totally, sometimes progressively. This permits living systems to accumulate information over generations, but has the drawback, in multicellular organisms, to open the door for immortalization, leading to cancer.

A phylogenetic analysis of bacterial genomes shows them to comprise persistent genes, the <<paleome>> (Greek: palaios, ancient, reminiscent of the origin of life), associated with genes permitting development of life in a particular niche, the [symbol] cenome [symbol] (from koinos, common, a radical often used in ecology).

Most ribonucleases belong to the former, demonstrating their central position in core life processes.

These enzymes appear to have often (but not always) evolved through consistent scenarios, generally grouping bacteria into well-defined clades.

The evolution of phosphorylases (which salvage energy) is particularly revealing, resulting in diverse complex structures whose function is to degrade RNA.

The degradosome of the gamma-Proteobacteria is a paradigm of such complex structures that emphasizes the essential role of energy in degradative processes.

The A+T-rich Firmicutes behave in a highly original manner, where many ribonucleases and related proteins co-evolve as a group.

The recent dentification of novel activities in these organisms, stresses the (underestimated) importance of degradation of very short RNAs, as well as 5′ to 3′ degradative processes in Bacteria.

### BRIEF DESCRIPTION OF THE DRAWINGS

FIG. 1.

### A.

Organization of Bacterial Genomes Illustrated by the Conserved Clustering of Genes in Pseudomonas putida KT2440.

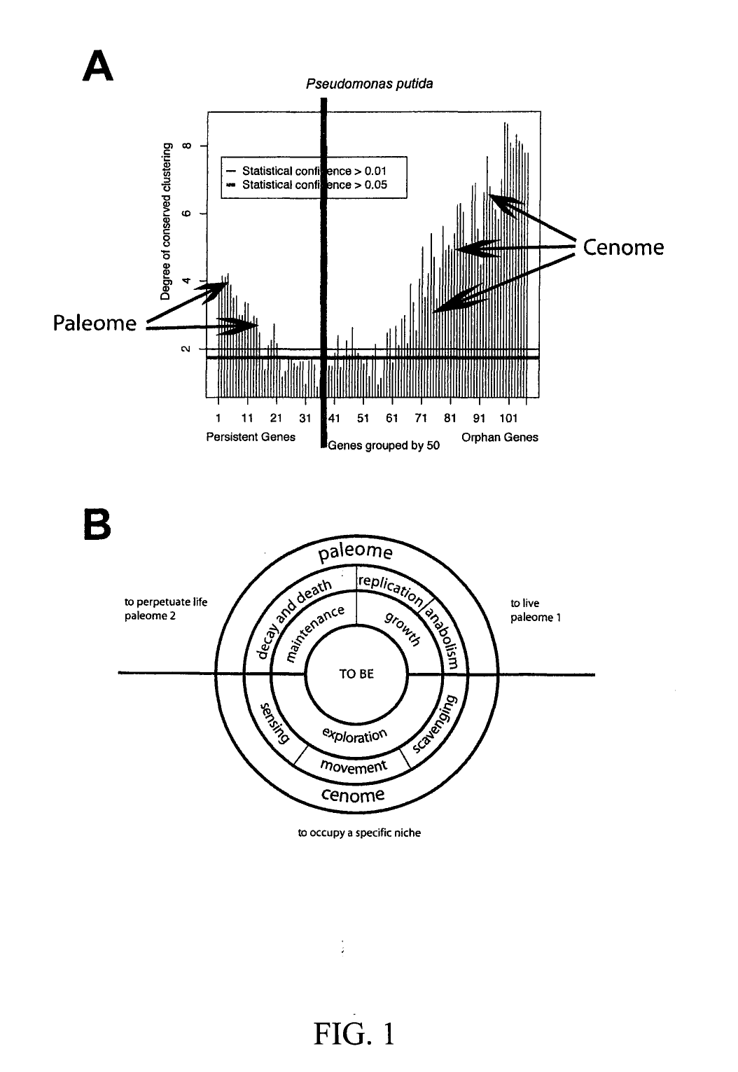

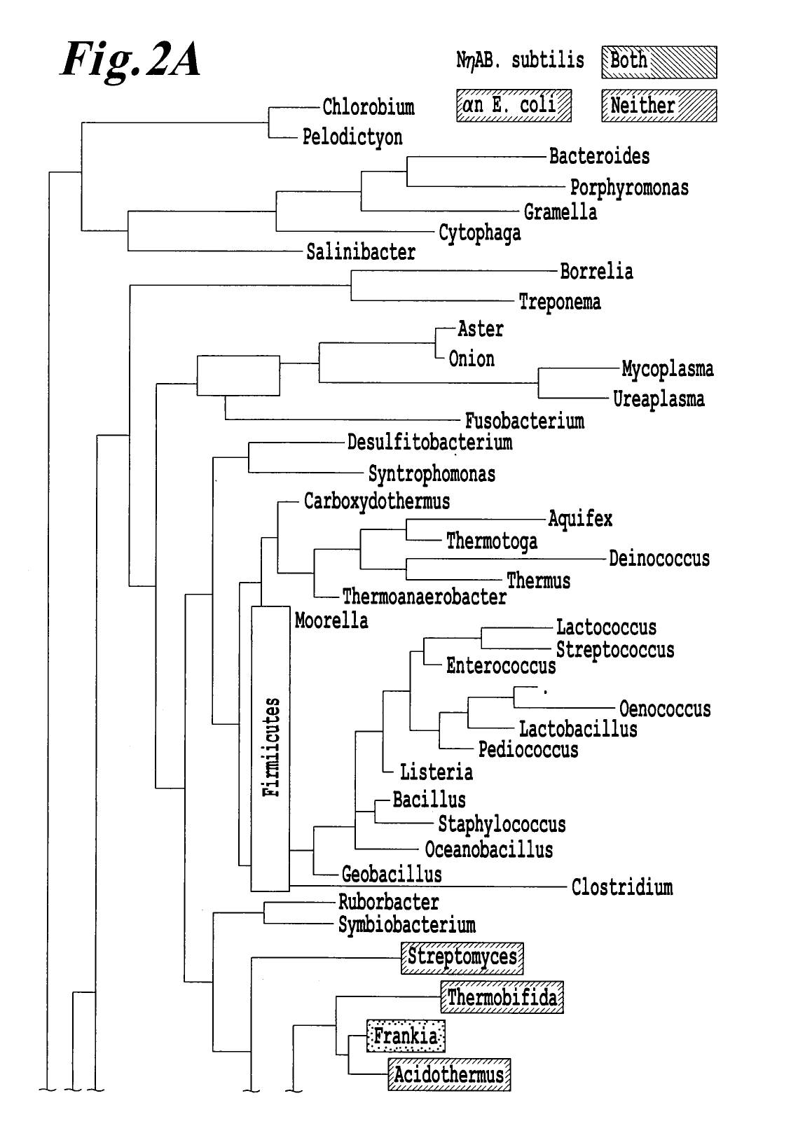

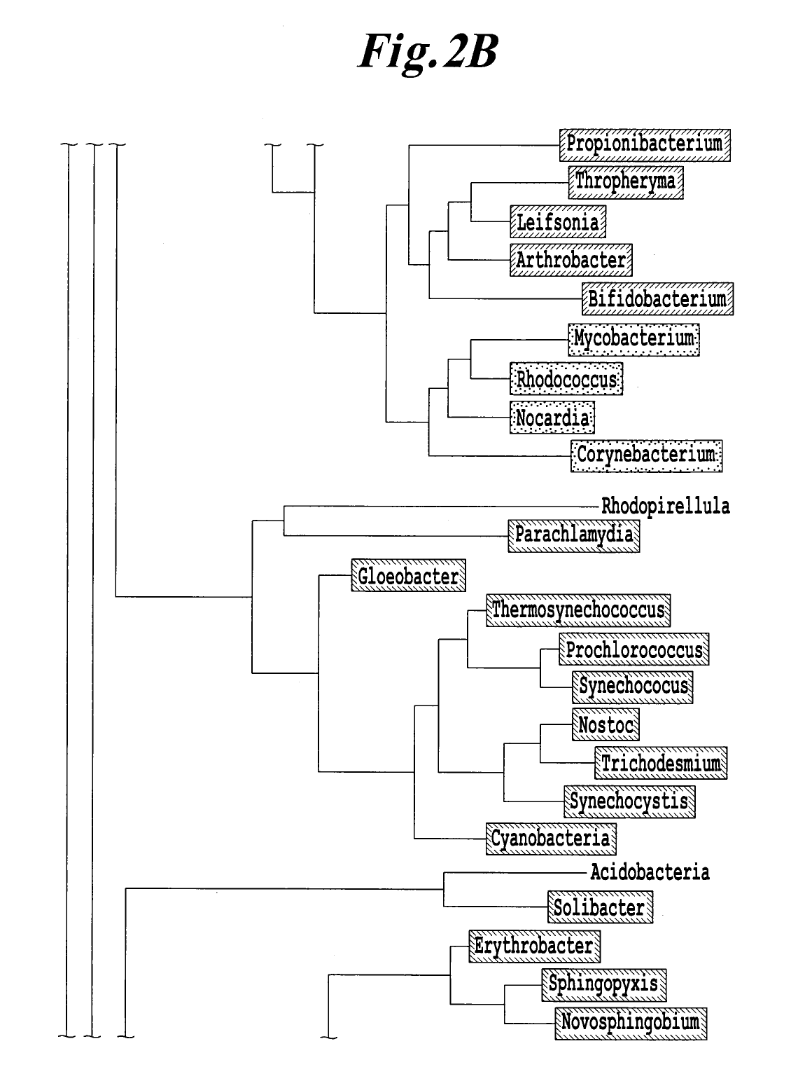

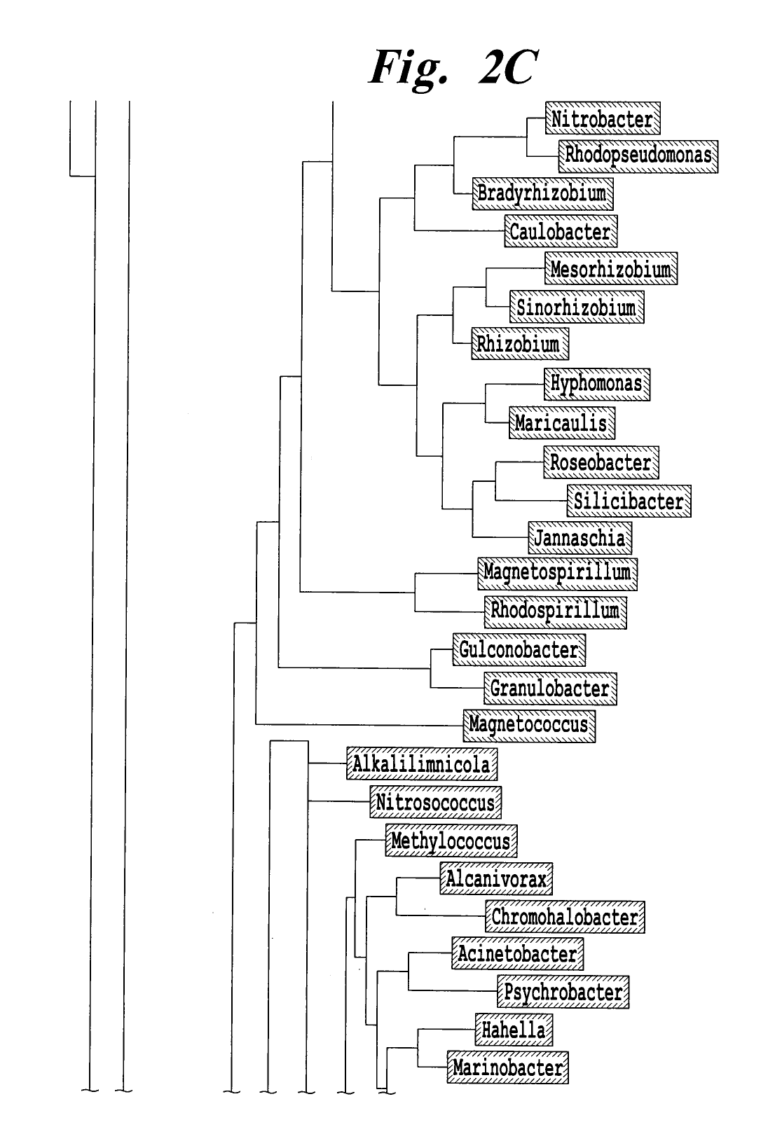

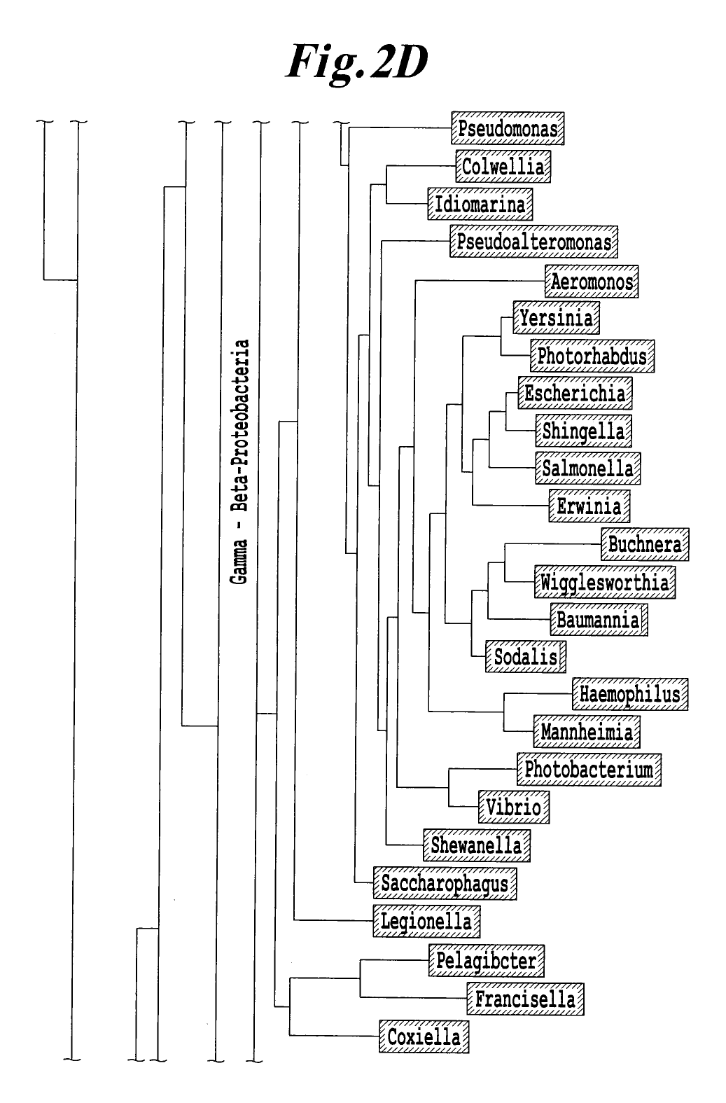

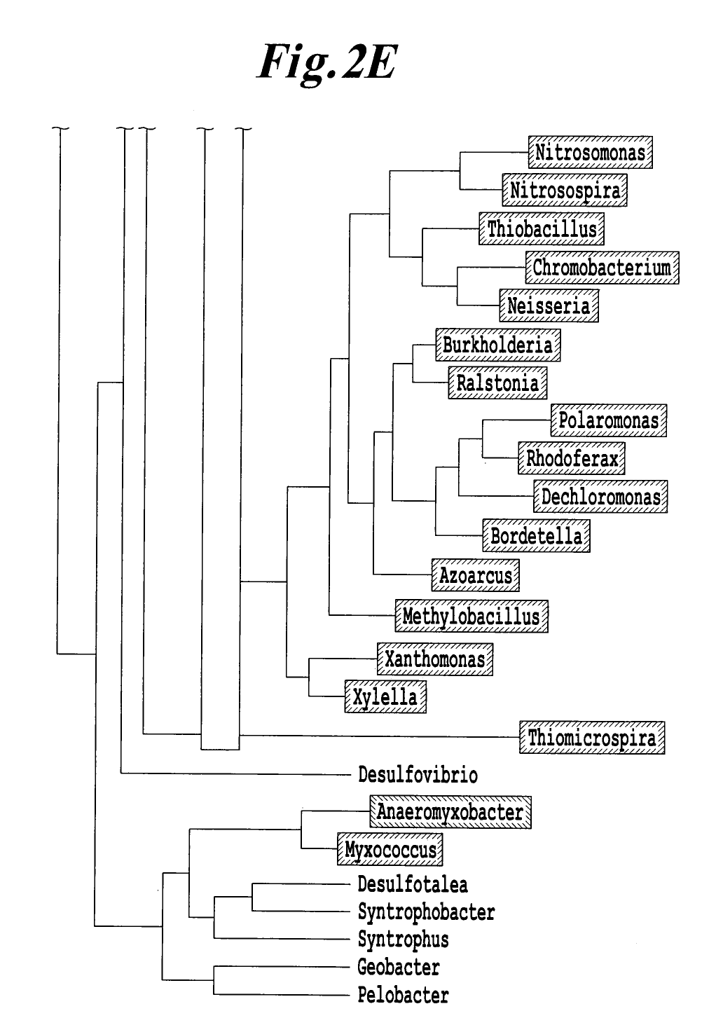

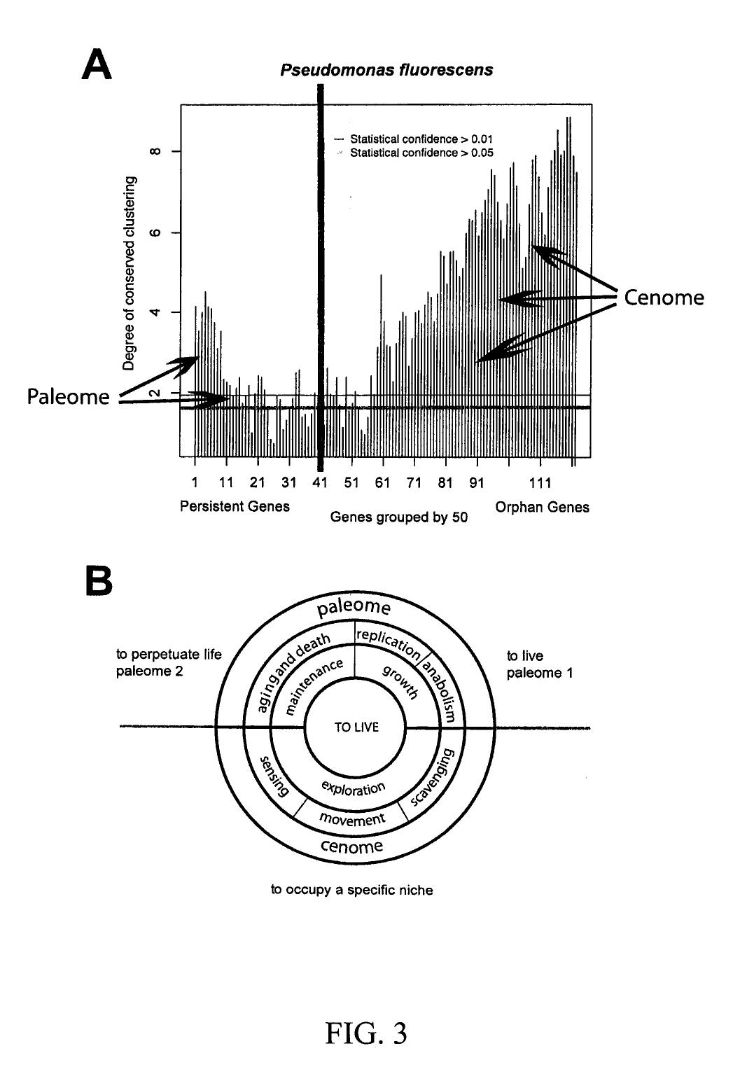

Genes have been grouped together into groups of 50 genes as a function of their frequency in more than 150 bacterial genomes comprising more than 1,500 genes (horizontal axis) and their tendency to remain clustered in many genomes is plotted on the vertical axis (drawn after a panel of Supplementary FIG. 1 in reference (13)).

The horizontal line spanning the figure gives the statistical validation of this tendency for genes to remain clustered (p-value <0.01 and p-value <0.05).

Genes that tend to be present in all genomes tend to remain clustered, while rare genes are also clustered together.

This clustering process stems from completely different reasons as demonstrated in (15).

The former class constitutes the paleome, while the latter makes up the cenome (196).

### B.

Genes Constituting the Paleome and the Cenome Code for Functions that Display Consistent Roles in the General Processes Defining Life.

The paleome is formed of two major parts.

Paleome 1 makes life itself, coding for anabolism and replication, while Paleome 2 codes for maintenance functions that are required to perpetuate life (69).

Most RNases are distributed among both parts of the paleome.

The cenome comprises the rarer RNases that are used to scavenge RNA from the environment and perform all functions that would be useful for exploration, as well as those involved in managing horizontally transferred elements.

FIG. 2.

Distribution of nanoRNases in the Bacteria Phylogenetic Tree (Drawn after Supplementary FIG. 1 from Reference (179)).

Firmicutes as well as bacteria growing at high temperature (Aquificales and Thermotogales) have the NrnA counterpart, while beta- and gamma-Proteobacteria have Orn.

Actinobacteria appear to have both.

Cyanobacteria and alpha-Proteobacteria have none, asking for identification of the protein structure performing the corresponding activity.

FIG. 3. Gene Clustering in a Bacterium with a Large Genome, Pseudomonas fluorescens, as a Function of their Frequency in Bacterial Genomes.

A. On the abscissa, genes are grouped by clusters of 50 genes, as a function of their frequency in available bacterial genomes longer than 1,500 genes: the leftmost groups are present in most if not all genomes, and this number progressively decreases along the abscissa, with the group of 50 genes present on the rightmost bars present only in P. fluorescens. On the ordinate is represented the tendency for the genes in each group of 50 genes to remain clustered together in the genomes where they are present. The grouping of the genes on the left, making the paleome, is reminiscent of a scenario of the origin of life, while the genes on the right, making the cenome, permit cells to occupy a particular niche (Danchin 2007).

B. The genes in the paleome make two categories, persistent essential genes and persistent nonessential genes (Fang et al 2005). The latter category codes for proteins that use energy to maintain and repair the cell functions as well as genes involved in managing energy involving polyphosphates.

### DETAILED DESCRIPTION OF THE PREFERRED EMBODIMENTS

Based on the discussion provided herein, the present application provides a method of assessing the ageing process of a cell lineage, in which

assessing flux and/or rigidity of at least one member selected from the group consisting of metabolic pathway, conformation of a protein (proline isomer), absence of isoaspartates in a protein, degradation of mRNA, tRNA, and/or rRNA, and activities of one or more of RNase, RTP, polyphosphorylases, Ribonuclease PH, poly(A) polymerase, polynucleotide phosphorylase, ATP-dependent RNA helicases, polyphosphate synthase, polyphosphate kinase, polyphosphate enolase, ribonuclease G, ribonuclease E, Ribonuclease M5, Metallo-β-lactamses, RNase Z, RnjA/B, ribonuclease III, ribonuclease MrnC, ribonuclease H1, ribonuclease HII, ribonuclease HIII, ribonuclease II, ribonuclease R, ribonuclease D, ribonuclease T, mRNA 5′-pyrophosphatase, nanoRNases, and combinations of these in a cell lineage at a first time point,

assessing flux and/or rigidity of at least one metabolic pathway, conformation of a protein (proline isomer), absence of isoaspartates in a protein, RNA degradation of mRNA, tRNA, and/or rRNA, and/or one or more of the activities in a cell lineage at a second time point and/or a time point subsequent to the first time point, and

correlating a change of those assessed items in a cell lineage at a second time point and/or a time point subsequent to the first time point relative to the first time point, a change being indicative of cellular ageing.

Other embodiments of this method can employ one or more of the enzymes, genes and/or activities described herein. All of these pathways, enzymatic activities, genes, etc. are known in the art, i.e., their structures and functions are known. Therefore, assessing these indicia, whether it be the nature of a pathway, enzymatic activity or sequence of a gene are within the knowledge available to the person in this field. See, for example, the databases maintained by the National Center for Biotechnology Information at the National Library of Medicine and National Institutes of Health (U.S.A.).

For example, some of the pathways embodied by the present invention include: Glycolysis, Anaerobic respiration, Krebs cycle/Citric acid cycle, Oxidative phosphorylation, Fatty acid oxidation (β-oxidation), Gluconeogenesis, HMG-CoA reductase pathway (isoprene prenylation chains, see cholesterol), Pentose phosphate pathway (hexose monophosphate shunt), Porphyrin synthesis (or heme synthesis) pathway, Urea cycle, Photosynthesis (plants, algae, cyanobacteria), Chemosynthesis (some bacteria), glucoronate metabolism, lactose and galactose metabolism, inositol metabolism, cellulose and sucrose metabolism, starch and glycogen metabolism, glycine serine and alanine metabolism, valine, threonine, leucine and isoleucine metabolism, purine biosynthesis, histidine metabolism, pyrimidine biosynthesis, glutamate amino acid group synthesis, fermentation, electron transport and others. See also, e.g., Shimizu, A review on metabolic pathway analysis with emphasis on isotope labeling approach Biotechnology and Bioprocess Engineering Volume 7, Number 5/October, 2002; Regulation of primary metabolic pathways in plants, By Nicholas J. Kruger, Steven A. Hill, R. George Ratcliffe, Phytochemical Society of Europe. These pathways and genes/proteins associated therewith are known in the art from databases such as the BioCyc database collection and the databases maintained by the National Center for Biotechnology Information at the National Library of Medicine and National Institutes of Health (U.S.A.).

The types of cell linease to which this methodology can be employed are preferably prokaryotic or eukaryotic cell lineages. Each of these classes of cells or organisms are well-defined phylogentically and each have characteristics which place them into those classifications.

Examples of prokaryotic cells can be found, for example, in Biology of Microorganisms, Eleventh Edition, by Madigan, Martinko and Parker Prentice Hall (2005).

Examples of eukarotic cells, include but not limited to yeasts (saccharomyces) and mammals such as dogs, cats, horses, pigs, sheep, goats, chickens, fish, and humans.

This method can be used to asses how cancer cells form and for a means to target cancer cells in new ways.

In another aspect of the invention, a method of identifying an agent that effects the biological ageing process of a cell lineage is provided. This method includes

assessing flux and/or rigidity of at least one member selected from the group consisting of metabolic pathway, conformation of a protein (proline isomer), absence of isoaspartates in a protein, degradation of mRNA, tRNA, and/or rRNA, and activities of one or more of RNase, RTP, polyphosphorylases, Ribonuclease PH, poly(A) polymerase, polynucleotide phosphorylase, ATP-dependent RNA helicases, polyphosphate synthase, polyphosphate kinase, polyphosphate enolase, ribonuclease G, ribonuclease E, Ribonuclease M5, Metallo-β-lactamses, RNase Z, RnjA/B, ribonuclease III, ribonuclease MrnC, ribonuclease H1, ribonuclease HII, ribonuclease HIII, ribonuclease II, ribonuclease R, ribonuclease D, ribonuclease T, mRNA 5′-pyrophosphatase, nanoRNases, and combinations of these in a cell lineage at a first time point,

contacting the cells of the cell lineage after the first time point with the agent,

assessing flux and/or rigidity of at least one metabolic pathway, conformation of a protein (proline isomer), absence of isoaspartates in a protein, RNA degradation of mRNA, tRNA, and/or rRNA, and/or one or more of the activities in a cell lineage subsequent to said contacting, and

correlating a change of those assessed items in a cell lineage at the subsequent time point relative to the cells in the cell lineage that have not been contacted, a change being indicative that the agent effects cellular ageing.

The agents can be, for example, biological molecules (e.g., RNA, DNA, protein, peptide, amino acids, lipids, carbohydrates, antibodies, antibody fragments (e.g., F'ab fragments), derivatives of these, and synthetic forms of these), other polymeric materials (natural or synthetic), organic molecules (e.g., containing phenyl ring(s), pyridine ring(s), pyrimidine ring(s), 5-12 membered rings such as heterocycles having S, O, N, halogens, etc, fused ring structures), and also inorganic materials, e.g., salt forms etc.

### Information and Natural Selection

### Creation of Information is Reversible

Information is a deep concept, typical of what the mathematician John Myhill named a <<prospective character>>, i.e. a concept that becomes deeper and deeper as it is further discussed (Myhill 1952). Since its first formalization in the theory of communication devised by Shannon (Shannon and Weaver 1949), the depth of the concept has been progressively increased in the case of sequences of symbols, typical of what we find in genomes (Cover and Thomas 1991; Yockey 1992; Danchin 2003). For a long time it was intuitively assumed that creation of information required energy (typically, according to Leo Szilard, creation of one bit of information would require ½ kT in energy (Szilard 1929)). As a natural outcome of this intuition, Rolf Landauer, working at IBM on the integration of logical gates into what was to become microprocessors, asked the question whether this would lead to unbearable heat in computing devices, which were meant to become smaller and smaller, while computation kept becoming faster and faster as technology kept improving. In a seminal article in 1961, curiously overlooked, he showed that creation of information, contrary to the accepted view of the time, was reversible and therefore did not involve energy consumption (Landauer 1961). Charles Bennett later completed the theory. The overall outcome was that creation of information is reversible, but that erasing information from the memory where all the intermediary steps are collected requires energy, typically of the amount predicted by Szilard (Bennett 1988). In short, creation of information can occur in physical systems, and does not contradict any principle in physics. However Landauer and Bennett remarked that information is created among a large number of states that occupy a huge number of space or energy states. When considering the production of information, therefore, the question is not whether it is possible, but how it can be recovered from an ocean of non informative states.

With this understanding, evolution of living organisms (and of any other physico-chemical process where there is accumulation of information) can be understood as the question of telling which, among a variety of individual elements at a given time, must be retained for further evolution. Put otherwise: considering creation of information as an authentic principle of physics, what is the associated principle that makes emerge the entities that have been accumulating information? Because this process requires telling some entities from the bulk, it is a screening process. Because this process requires making room for these entities to accumulate, it is a selective process. Hence, the physics of any system producing information cannot work without a process of selection. In a nutshell, presented in this way, Natural Selection is an authentic principle of physics.

### Natural Selection as a Novel but Straightforward Principle of Physics

This being said—and this rests on the demonstration by Landauer and Bennett that creation of information is reversible—it becomes essential to understand the principles of how Natural Selection—a concept of Biology—works in physical terms. Several ideas can be put forward. I develop here the main directions along which at least one pathway works, that seems to operate at many levels in biological systems, and we illustrate it in the case of bacteria. The basic idea is to follow the second part of the Landauer-Bennett theorem, that which states that erasing memory requires energy, and to look for processes that use energy in an intuitively unexpected way in order to make the relevant ones stand out.

Creation of information requires many steps, it starts from a given complex of dynamic interacting entities, that progressively transforms into variants, among which some have a higher information than that of the original complex. (Some of) those (these entities comprise physical objects, but also processes, such as metabolic fluxes) which are not relevant in terms of increase in information need to be destroyed to make room for those which have a higher information. Within this conceptual frame, this destruction process—Natural Selection—cannot simply be identified with ageing or weathering followed by decay. It must, at least from time to time, actively discriminate between entities that are in some degree functional and those that cannot function. This is because the process needs to avoid destroying the elements that carry an increased information, and this is where energy comes in. Energy has to be consumed to make innovations stand out, in a discriminant process: energy is used to prevent degradation of functional entities, permitting destruction of the non-functional ones (see below).

We note that with this view Natural Selection cannot be purely random or neutral (Kimura and Ota 1974). Because it requires energy to operate, it can retain or recruit functional entities: this explains part of the controversy surrounding the concept, which is usually assumed to be purely passive (see however the widely spread spencerian “selection of the fittest” (Spencer 1864), which assumes implicitly some mechanism permitting comparison between fit and unfit organisms). Deep implications of the present contention cannot be discussed in this short essay. It can be noticed however that Kimura himself is aware of the difficulties inherent to a purely neutral selection process, as he introduced an active selective parameter to take this requirement into account (Kimura 1979). Indeed, it can be shown that purely blind destruction would not work in the long term in a finite population because it would erase both uninformative and informative objects or processes. To understand Natural Selection we must therefore consider specifically the destructive (selective!) steps that are associated to energy consumption and identify, among these, those which are relevant to information.

Interestingly, and in agreement with this line of reasoning, many processes pertaining to improvement of the quality of information are pervasive in molecular biology processes. Noting that a straightforward application of the inevitable noise created by thermal fluctuations at 300 K (the temperature where living organisms thrive), would lead to an unbearable error rate in the synthesis of proteins, John Hopfield (Hopfield 1974) and Jacques Ninio (Ninio 1975) analysed the essential step of translation of messenger RNAs into proteins along lines similar to those that would draw aside the hypothesis of strict neutralism. They proposed the following idea to account for the remarkable accuracy of the translation process (deciphering the genetic code without an energy-requiring selective step would lead to an error rate resulting in synthesis of essentially aberrant proteins): there is proof-reading steps that tell whether a correct amino acid is loaded on tRNA, and a correct codon is deciphered, and that discard the erroneous object from their target interactions. These essential steps use energy, via ATP and GTP hydrolysis. The basic idea is simple, as a thought experiment illustrates (Gueron 1978): in a pool where we have both black and red fishes swimming together, we wish to catch (almost) exclusively red fishes, knowing that, when in a net with holes having a shape of the size of the fish, but asking for some effort to go through, black fish react very fast and succeed in getting out, while the red fishes are much slower in that escape. The idea is to use a net, catch fishes (equally black and red) and wait with the net suspended above the pool for some time (not too short, not too long), and then place it on the bank, recovering fishes: most will be red fishes. This is clearly a selective energy-consuming, information-gaining procedure.

This idea can be generalized as follows. An aged biological system expresses particular objects (RNAs, proteins, metabolites) and processes (metabolic pathways) some of which are accurate, some of which are variants. Despite this lack of exactitude, an aged system will generally be able to express metabolic pathways and generate young systems, thereby making new objects using old objects (Aguilaniu et al 2003). It needs however to be able to sort out those which are functioning best. We assume that this requires energy, noting that if this assumption is correct, then we need to identify the genes coding for the corresponding objects. In short, as implicitly recognized by Dyson when modeling the first steps of the origin of life (Dyson 1985), reproduction can increase the information carried by the system. This increase comes as individual steps. Accumulation of information requires a ratchet-like process that will progressively accumulate accurate objects in a collection of objects that, for that particular purpose, can be considered <<junk>>, but are not <<waste>>. This requires that room is made to permit this accumulation. Making room asks for destruction of some of the objects that occupy the corresponding space, but not in a strictly random way as accurate objects need to be preserved from destruction. Again, random destruction would destroy accurate and inaccurate objects equally well, and would not, in general, result in accumulation of accurate objects.

Hence, and this is the most important feature of the present conjecture, that differs from previously proposed models usually meant to propose scenarios for the origin of life, there is a need for a process that, using energy, selects out of the mixture those objects that should preferably not be destroyed. The difference between accurate and inaccurate entities is that the former can be engaged in forming complexes or in metabolic processes. We postulate that when this happens, the entity (process or object) has features—possibly a particular metabolic flux, possibly rigidity, possibly specific conformation features such as a specific proline isomer, absence of isoaspartates in proteins, etc—that can be identified by the degradative machinery. Using a source of energy these entities could be rejected by the machinery, and therefore remain intact. By contrast, entities that are not engaged in a complex or a metabolic process would be degraded. This is an exact counterpart of the Hopfield-Ninio kinetic proof-reading process in macromolecular biosyntheses.

### Comparative Bacterial Genome Organization

The proposed view assumes that information can be accumulated in a ratchet-like process separating between reproduction and replication. Until now it is presented in an abstract way. It is therefore essential to see whether we can identify in bacterial genomes the (presumably ubiquitous) functions that would explicitly be related to these processes. More precisely, we need to identify among those the functions which would code for the energy-dependent processes we have postulated. In this endeavour we benefit from the huge number of genome sequences that are now available. However, a practical difficulty prevents the obvious straightforward comparison between all genomes, looking for genes which would be ubiquitous. Indeed, functions can be fulfilled by different objects and ubiquitous functions are often expressed via gene products of different origins: acquisitive evolution is pervasive in biology (Thompson and Krawiec 1983; Ashida et al 2005). Hence, even if we expect some functions to be ubiquitous, we do not expect that the genes of interest will be strictly ubiquitous. Fortunately, because organisms derived from each other via an evolutionary process, when an object has been selected in performing a function, it tends to persist through generations, allowing us to identify “persistent” genes, i.e. genes that are present in a clique of genomes but not necessarily in all of them (Fang et al 2005). Subsequently, knowing these genes permits us to identify (most of) the ubiquitous functions we need to consider as essential to life and to identify the corresponding genes when they do not belong to the persistent set (see an example in (Mechold et al 2007)).

Analysis of a large number of bacterial genomes allowed us to construct a set of persistent genes and of their conserved syntenies. A detailed analysis of conservation of proximity of genes in genomes revealed a remarkable feature of their organisation: both persistent genes and rare genes tend to stay clustered together (Danchin et al 2007), making two highly consistent families of genes, separated by a large twilight zone (FIG. 1). Remarkably, the connection network of the persistent genes is reminiscent of a scenario of the origin of life, suggesting “paleome” (from παλαιo [symbol] , ancient) for its name, as it recapitulates the three phases of a scenario based on surface metabolism: synthesis of small molecules on solid surfaces (including ribonucleotides and co-enzymes), substitution of solid particle surfaces by an RNA-world where transfer RNA played a central role, and invention of template-mediated information transfer (Danchin 1989). The paleome is made of approximately 500 genes, that both tend to persist in genomes and persist in the way they cluster in genomes.

This gene set is made of two approximately equivalent subsets, that differ in the way they can be inactivated with or without loss of capacity to result in colony formation on plates. Essential genes (which cannot be inactivated without loss of capacity to live) contribute to the construction of the cell and to replication of its genome. They make approximately half of the paleome. The complement is made of non-essential persistent genes. They are involved in functions that are generally annotated as related to maintenance and repair as well as to some specific metabolic pathways (Fang et al 2005). This latter class is not strictly essential, as the corresponding genes can be inactivated without total loss of viability (Kobayashi et al 2003; Baba et al 2006). Analysis of the paleome therefore suggests that we should separate between two different processes, both essential in the long term but with different contribution to essentiality in the short term: the latter category of genes appears to contribute to perpetuation of life rather than to permit life per se. Preliminary experiments are indeed consistent with this view: the plating efficiency of strains inactivated in genes of the second category is rapidly decreasing over time.

By contrast, the set of genes acquired by horizontal gene transfer corresponded to genes important for the cell to survive in a particular environmental niche, not to provide the basal functions for life. This very large class, which we do not consider further here, tends to comprise new members in different strains of the same species. It has accordingly been named the cenome (Danchin 2007), to make reference to its role in permitting the organism to live in a particular niche (after κoινo [symbol] , common, biocenose is a common concept in ecology, created by Karl Möbius in 1877, see e.g. (Movila et al 2006)).

### Producing New Information Via Energy-Dependent Degradation Processes

The information accumulating process I considered has to be very effective, and to result in an information-rich outcome in a fairly large number of situations. To understand selective stabilization of novel information during reproduction, we must therefore consider specific destructive steps that are associated to an energy-consuming step and identify, among these, those which are relevant. This implies that specific processes are implemented to put into action the metabolic capacity of the organism. A central question we need to answer is identification of core metabolic processes that are used to permit selection of young structures in an aged organism. Can we find them into the non-essential part of the paleome?

At this point of our reasoning we find that we possess two elements in our conjecture which may be related to each other and may help us to identify the missing elements. On the one hand we need to find out genes that correspond to the energy-dependent processes necessary to accumulate information in a ratchet-like mechanism during the reproduction phase of the life of the organism. On the other hand we have a set of persistent genes, corresponding apparently to ubiquitous functions (for example, this is the case of the degradation of very short RNAs, an essential step coded by different proteins in different bacterial clades (Mechold et al 2007)), to which we could not easily ascribe an essential role in the cell.

An analysis of this latter class of genes shows us that, indeed, energy is involved in many of their functions. In particular we find that many of the enzymes of the “degradosome”, essential for RNA degradation, belong to this paleome genes category (for example, the core of the degradosome, polynucleotide phosphorylase, which uses phosphorolysis to produce nucleoside diphosphates—energy-rich compounds—is indeed non essential (Portier 1980)). While this structure has explicitly been identified in E. coli, where it is associated to polynucleotide phosphorylase and enolase, both energy compliant functions (Carpousis 2007), the counterpart has not yet been identified whether in Firmicutes or in Eukarya, for example. However a thorough review of the literature, associated to a general analysis of the co-evolution of genes in B. subtilis suggests that, at least in the former case, a degradosome-like structure, directly associated to energy-producing enzymes of glycolysis, is also likely to exist in Firmicutes (Danchin 2008). In the case of Eukarya, finally, the exosome is also tightly associated to RNA phosphorolysis, not hydrolysis, which is an energy-saving process (Lin-Chao et al 2007). In parallel, genes coding for ATP-dependent proteases as well as ATP-dependent RNA helicases belong to the paleome. Taken together these functional associations are remarkably consistent with the present conjecture.

A central question remains, however. That of the origin of the energy source that will be used for the information preserving processes. Here again, analysis of the paleome genes provide us with an extremely interesting hypothesis. Indeed, among non-essential paleome genes are the genes that metabolize polyphosphate (poly(P)). Generally assumed to play the role of phosphate storage, poly(P), a ubiquitous potential source of energy, is systematically associated to RNA degradation and in particular to the E. coli degradosome (Blum et al 1997), as well as to the predicted homologous structure found in Firmicutes (Danchin 2008). Poly(P) has been detected in various amounts in all organisms tested to-date (Brown and Kornberg 2004). The detailed pathways of its metabolism are however still very poorly known, despite the extensive work of the late Arthur Kornberg and his co-workers who unraveled many of the remarkable features of poly(P)-deficient strains (Fraley et al 2007). Three poly(P) biosynthetic pathways are known, but they apparently fail to account for the bulk of poly(P) accumulation. The major enzyme of poly(P) metabolism, polyphosphate kinase (PPK) catalyzes reversibly and processively phospho-transfer between ATP and poly(P). Another class of processive polyphosphate kinases, PPK2, prefers GTP and is widely conserved in Bacteria (Zhang et al 2002). Many organisms have both PPK and PPK2 in their genomes, a few have only PPK2, some only PPK, and many have neither PPK nor PPK2. The latter have however enzymes, namely PpnKA (YjbN) and PpnKB (YtdI), involved in the poly(P)-dependent biosynthesis of NADP (the activity has been biochemically established in Mycobacterium tuberculosis (Raffaelli et al 2004)) which could be coding for the missing (or a subunit of the missing) polyphosphate kinase. In B. subtilis ppnKA is an essential gene in synteny with several genes involved in RNA metabolism or related activities (Danchin 2008).

When cells are ageing, under circumstances when nutrient supplies are limiting, for example, the energy-charge of the cell will be depleted. In particular it cannot be expected that ATP is stable enough to maintain its concentration over a long period of time. Under such circumstances a mineral would be particularly stable, and this may well account for the ubiquity of poly(P). The role of this mineral has not yet been explored in this light, although Brown and Kornberg proposed that it might have had an important role at the origin of life (Brown and Kornberg 2004). Furthermore, it seems remarkable that some of its properties would be extremely fit with the present conjecture. Indeed, for example, some biochemical assays of adenylate kinase can use poly(P) as the energy-rich phosphate bond instead of ATP (Ishige and Noguchi 2001). The second step in the formation of nucleoside triphosphates involves nucleotide diphosphokinase: it can also use poly(P) as a energy-rich phosphate donor, restoring easily the ATP and GTP pools (Ishige et al 2002). Associated to the phosphorolysis of RNA (Danchin 2008), it seems therefore quite likely that an aged cell will be able to restore its energy capacity well before it can make its core metabolism based on electron transfers work. In this respect it may also be revealing that NAD kinase may use poly(P) as a phosphate donor (Raffaelli et al 2004), as NADP is the major electron transfer coenzyme directly associated to anabolism, i.e. construction of the cell. One of the intent of this short essay is to convince investigators that it might be important and extremely interesting to explore further this putative role of poly(P).

Finally, I can venture to propose a further exploratory conjecture. Poly(P), by its very structure, is a strong calcium binding mineral. It is present in all cells, and if it has the role we attribute to it, it may also have recruited other functions, as this is very frequently witnessed in evolution. An obvious one, because it is a mineral, is that it could play a role in the structural architecture, the scaffold or casing of organisms. This may have been at the origin of bones in vertebrates. Why not think, then, that bones could still have some of the initial function of poly(P) as an energy source used against ageing processes? This might even be at the root of osteoporosis, linking the present conjecture about ageing in bacteria with ageing in vertebrates. Only deep epidemiological studies associated to genetic studies may tell whether this is a valid conjecture or sheer nonsense (Livshits 2005) . . . .

### Provisional Conclusion

Often taking these processes as interchangeable (and using both words as equivalent), general reflections on life or ageing have often favoured to emphasize replication, not reproduction in the process of cell multiplication (Szathmary 2000; 2006; Brosh and Bohr 2007; Burhans and Weinberger 2007). This led to a particular interpretation of the important processes of maintenance and repair, which certainly play a major role in sustaining the life of individuals as meant to preserve as long as possible their integrity. In the case of higher organisms, deeper reflections however, in particular those on the disposable soma theory (Kirkwood and Holliday 1979) and on the evolution of life histories (Stearns 1992), emphasized the role of reproduction as separated from that of replication, and showed that the functions of maintenance and repair had to be conceptually separated from those involved in reproduction.

The most common views are based on the spencerian interpretation of darwinism as [symbol] selection of the fittest [symbol] , where maintenance and repair are systematically associated to competitive processes permitting extension of life span against fertility, as the limited amount of resources that can be devoted to counteract ageing, will jeopardize the same limited resources that can be devoted to fertility (Holliday 2006). Except in conditions where analyses deal with questions relating to the origin of life or with the error catastrophe, most of these reflections are based on ageing processes involving multicellular organisms. I have tried here, using the recent demonstration that bacteria age and therefore that aged entities must produce young ones, to explore the duality reproduction/replication and to relate it to the increasing knowledge of bacterial genome organization that we derive from comparative genomics.

This view, as the one that several decades ago proposed the disposable soma theory but following a different path, goes far beyond the molecular processes permitting maintenance and repair. It captures their essence along a further line, the fact that in many occasions they are poised to increase the information content of the entities (physical objects and dynamic processes) that are simply interpreted as being repaired. I have tried to show here that considering the remarkable properties of information management in material systems permits us to consider living organisms as poised to accumulate information in a ratchet-like manner. As in the disposable soma theory, the ultimate outcome of this process is to escape the inevitability of ageing, not by perpetuating the individual but by systematically creating a progeny made of young organisms. In some instances, this happens by accumulating aged structures in the mother cell, as in Saccharomyces cerevisiae (Aguilaniu et al 2003). In other cases, such as rapidly dividing bacteria, the aged structures concentrate at the poles, progressively leading to aged cells which will die, and young cells which will continue multiplying (Lindner et al 2008). Even in multicellular organisms, where it was thought that mitosis resulted in identical daughter cells, it is now established that proteins specifically targeted for proteasomal degradation are inherited preferentially by one mitotic daughter during somatic cell division (Fuentealba et al 2008).

This process, which accumulates information in at least one member of the cell's progeny has the considerable advantage that, at the same time, it is setting the stage for accumulation of any type of information that may have been created. The consequence is that this may occasionally result in evolution of progressively information-richer cells. In this context it becomes interesting to entirely reappraise the discovery of “adaptive mutations”, which caused much controversy twenty years ago because they were (wrongly) interpreted as suggesting lamarckian evolution (Cairns et al 1988; Danchin 1988). Indeed, the core of the reflection proposed here is the idea that, in order to accumulate information, there is a need for a process of <<making room>> for young or novel entities that will replace the used or aged ones. It could well be, then, that at least some of the genes of the non-essential paleomes are involved in this process. We have constructed a particular E. coli strain that permits easy identification of adaptive mutations (Danchin 1993 (2007)), and we are in the process of investigating whether inactivation of some of the relevant genes prevents their creation. Success in this domain would be a significant proof of concept of the conjecture proposed here, where life and perpetuation of life being considered as separate processes, the latter being able to capture innovation.

Basing the reasoning on Information Theory, I see Natural Selection as this very process, which therefore becomes an authentic principle of physics. To my view, this is particularly remarkable, and may not be a coincidence (historically, related ideas tend to appear simultaneously everywhere), at a moment when classical physics and quantum physics are getting reconciled via a renewed view of Information Theory. As noted by Steane: [symbol] Historically, much of fundamental physics has been concerned with discovering the fundamental particles of nature and the equations which describe their motions and interactions. It now appears that a different programme may be equally important: to discover the ways that nature allows, and prevents, information to be expressed and manipulated, rather than particles to move [symbol] (Steane 1998). I wish to extend this view to the realm of Biology.

As a follow up of the observation that replication could not exist at the very origin of life as this would have required some kind of external intervention (Dyson 1985; Szathmary 2006), I have explored here, in the case of bacteria, the consequences of a reproduction process that would be able to create and retain newly created information, in particular. I have established that a system submitted to the trio variation/selection/amplification can accumulate information (within material objects or dynamic processes) when the selection process consumes energy in a step discriminating between stable and less stable interactions. Rather than simply derive a conceptual justification, as this has been repeatedly proposed under a variety of philosophical postulates (see one example of a recent discussion in (Sterelny 2001)), I suggested a concrete justification that may be submitted to experimental falsification. Because it is based on the <<measurement>> of the quality of an interaction (via its stability) this process will progressively result in the construction of systems accumulating interactions, typical of what is witnessed in the evolution of living organisms. A noteworthy consequence of this observation is that it is not a paradox that there is a general tendency for some branches of life to evolve, without predictable directions, toward progressively more elaborate forms (discovery of multicellularity was one of those remarkable events). We may further note that this process spans domains of physics and chemistry much larger than simply the extant living organisms, and that it may apply, with appropriate qualification, to the question of the origin of life in a view that complements and renovates Dyson's reflection.

At this point of the analysis, emphasis is placed on ageing, via the fact that reproducing metabolic processes can rejuvenate organisms via a selective catabolism that makes room for young functional objects by destructing old ones without affecting functional ones. It is therefore a reflection that is distinct from the prevalent view that ageing is a nonadaptive process that escapes the force of Natural Selection. Or, rather, this view would tend to separate the consequences of ageing in two different categories. In some cases, such as the situation illustrated in the disposable soma theory (which is valid not only for multicellular organisms but also for budding yeast) the aged individual plays the role of the rubbish bin, that collects non-functional entities and all innovation is in the progeny (when it exists). In other cases, as in pseudo-symmetrically dividing cells, the separation between aged and young entities is much less perfect and leads to progressive loss of viability (Stewart et al 2005). In this case there is much room for capturing innovation, as this can happen in the course of multiple generations. We conjecture that this might be the explanation of the existence of “adaptive mutation” repeatedly discovered in bacteria. In multicellular organisms, this process is typically developing at mitosis (Fuentealba et al 2008). This has the remarkable consequence that a phenomenon similar to that of adaptive mutations could permit stem cells to uncover pathways leading to their immortalization. The implementation of the process we conjecture requires an integrated ensemble of objects, typically those coded by the persistent gene set in bacterial genomes, the paleome. I have identified some of the relevant genes of the paleome and conjectured that a major source of energy involved in the maintenance system could be polyphosphate, a mineral that is ubiquitous in living cells. At this point of the reasoning we understand how an old cell, using energy, can renovate its machinery. A strong prediction of the present model is that the onset of the replication process should be delayed during this renovation step. Indeed, by contrast with reproduction, replication is strongly sensitive to the error catastrophe because of the recursive way it is put into action (Orgel 1963). It will be interesting to identify the counterparts of the paleome genes in eukaryotes, and to see how their role (including metabolism of polyphosphate) could be related to the onset of cancer.

### Functions for Life

The degradation of cellular components is usually viewed as part of a [symbol] cleaning [symbol] function, a maturation function, or as part of a regulatory cascade.

In parallel, as can be seen from the huge amount of effort devoted to the study of transcription factors, chromatin structure and regulatory cascades at the transcription initiation level, the paradigm for the control of gene expression at this level implicitly assumes that mRNA molecules will turnover, and, in Bacteria, turnover fast (1).

Curiously, however, the exact contribution of the degradation process to the ultimate level of the products of gene expression, namely proteins, has not been analyzed systematically.

And despite widespread observations of discrepancies between mRNA levels and proteins levels in Bacteria, the role of RNA degradation processes in the control of gene expression has, to say the least, been overlooked.

[symbol] Nothing in biology makes sense except in the light of evolution [symbol] (2) and the multiple roles of ribonucleases (RNases) in Bacteria should be explored in this light.

Condon and Putzer analyzed the phylogenetic distribution (3).

Rather than take the same approach here, which would simply update their thorough work, we take a different stance: a functional view of RNases considered from the stand-point of both the organization of the bacterial genome and the organization of metabolism.

In this review, we will use observations based on the study of evolution to try to convey the message that degradative processes are specifically involved in essential steps that are not simply meant to support life, but required to perpetuate life.

Before proceeding, a cautionary word is necessary.

The variation/selection/amplification of codon triplets that drives evolution continuously provides niches for particular new functions to be invented.

These functions are performed by chemical objects, which must be either recruited from previously existing objects (4; 5) or created de novo.

As a consequence, and this is essential for the discussion that follows, there is not a one-toone relationship between protein structure and function, so that, while many functions may be essential, and therefore ubiquitous, this does not have to be the case for the protein structures that support them.

This means that our view of phylogeny is not necessarily one that stems from a unique origin, e.g. LUCA.

Particular circumstances that led to the invention or capture of a function will be borne witness in phylogenetic trees with different shapes, related to the various scenarios for the origin and evolution of life that we may be tempted to consider.

However, because living organisms always derive from close ancestors, there is a tendency for organisms to stick to one object when it fulfills a given function.

Hence, it is most likely that the structure/function relationship will hold within a particular clade.

From time to time a jump, a discontinuity, will be observed, corresponding to the moment when a particular object was replaced by a new one.

In extant organisms, we do not expect to see many situations where two objects with the same function are found in a single species.

This is the famous “missing link” often observed in paleontology (i.e. the missing organism establishing the transition between two very different forms).

In this context, we will try in some cases to separate between a function (a generic answer to a general need, for example doublestranded RNA degradation) and its corresponding functionalities (features associated with the function which provide the closest response to a specific need, e.g. tagging for activity/inactivity, localisation in the cell, specification of a function to a narrow range of substrates, etc), trying to avoid making a direct connection between the connotations of the concept of function and that of evolution (i.e trying to avoid teleological interpretations: function “for” a particular purpose) (6).

Finally we need to add another, technical, caveat that must be taken into account.

Most of the data we analyze here are derived from genome sequencing projects.

These projects are experimental and are not error-free.

This implies that, while the presence of a sequence can be taken as firmly established, the same cannot be said for the absence of a sequence.

Before we draw important conclusions about a missing object, a very thorough investigation of the data leading to this conclusion is required.

In many cases, the absence of a sequence should therefore be taken with a grain of salt.

The concept of an RNA world at the onset of the evolution of life places RNA-centered functions at the core of the bacterial genome organisation.

However, this concept requires an earlier step, omitted from most scenarios, that led to the creation of nucleotides.

While the prebiotic soup scenario remains popular in the mass media, indepth analysis of the physico-chemical constraints for a medium in which life could be born supports a scenario where auto-catalytic reproduction of a surface metabolism would play a major role (7-11).

This scenario requires that surfaces be subsequently replaced by some sort of charged support.

The common hypothesis is that RNA played this role.

This implies that RNA was involved in the metabolic processes that led to, not only to its own synthesis, but also to that of amino acids, coenzymes and nucleotides.

Among extant RNAs involved in metabolism, transfer RNA (tRNA) has a special role.

Not only is it involved in ribosomal protein synthesis, but it is also involved in a variety of reactions that are unrelated to this process, including for example, synthesis of the heme precursor aminolevulinate, in a reaction that should not necessarily involve tRNA per se (11).

This makes ancestral tRNA a candidate of choice in the transition between the mineral world to the RNA world.

It is therefore expected that, very early on, tRNA was metabolized via ribozymes, typical of the enzymatic activities postulated to have developed in the RNA world.

Remarkably, the organization of bacterial genomes is consistent with this scenario of the origin of life.

Indeed, bacterial genomes are made up of two sets of genes, persistent genes, that tend to be shared by a majority of genomes (12), and genes that are highly variable and often present in only one strain of a given species.

Persistent genes make up a core structure that is organized into a network reminiscent of this scenario, forming the paleome.

Highly variable genes, directly related to the way bacteria occupy a particular niche, form the cenome, an unlimited collection of genes that are spread by horizontal gene transfer and (we do not have any indication yet about this possible process) sometimes perhaps created de novo (13) (FIG. 1).

The conservation of syntenies in genomes can be used to substantiate functional inferences, as the genes belonging to the paleome and the genes belonging to the cenome are often clustered together, albeit via completely different molecular processes (14; 15).

RNases belonging to the paleome are more directly involved in the basic functions involved in sustaining and perpetuating life.

The way they evolved will be discussed first in detail.

RNases (often secreted) belonging to the cenome, which provides functions permitting the cell to explore its environment and scavenge nutrients, will be discussed in a later section of this review.

Ribonucleases of the Paleome RNase P, a Ribozyme from the RNA World?

Evolution from a surface metabolism creating coenzymes, lipids and the basic building blocks (nucleotides in particular) to that of an RNA world suggests that the first macromolecular RNase activity could have been a ribozyme (16).

With this in mind, it is natural to begin the list of these essential functions with RNase P, a ribozyme that is involved in processing tRNA and other RNAs, riboswitches in particular (17; 18).

6 Ribonuclease P (RNase P) is the most widespread ribonuclease (3).

It is found in the vast majority of organisms so far examined (19).

Two exceptions have been documented, Aquifex aeolicus (3; 20; 21) and Nanoarchaeum equitans (22).

Although a recent article describes an RNase P-like activity in cell lysates of A. aeolicus, it has not been characterized and it is not yet possible to understand its relationship with known counterparts (23).

In N. equitans, tRNAs lack a 5′ leader and have therefore presumably been free to lose RNase P activity.

Other similar examples may be revealed with time.

RNase P is present within the subcellular compartments of eukaryotes known to synthesize tRNAs (24).

A key enzyme involved in the processing of tRNA, it is responsible for the generation of the 5′ termini of mature tRNA, via a specific endonucleolytic cleavage (19; 25).

In Bacteria, RNase P is a ribonucleoprotein consisting of a large catalytic RNA (P RNA, 350-400 nucleotides long) coded by the rnpB gene and a small protein subunit (approximately 13 kDa), encoded by the rnpA gene.

The RNA component of RNase P, which derives from a precursor processed by another ribonuclease, RNase E/G (26) (see below), is active in vitro in the absence of its protein subunit, but this activity is too slow to permit growth of RnpA-deficient strains (27).

This supports the idea that, during the transition from an RNA-world to the present biological world, proteins, with their superior catalytic properties, progressively invaded the catalytic functions of cells, both by acting as enzymes themselves and by stabilizing and enhancing the catalytic efficiency of ribozymes.

Two types of bacterial P-RNAs exist, based on sequence alignments associated with the 3D structure of the ribozyme: type A (found in Escherichia coli and Thermus thermophilus, for example) and type B (at present, found exclusively in Firmicutes) (28; 29), which are functionally interchangeable in vivo (30).

P-RNA contains two independent domains: the catalytic domain and the substrate binding domain (the Sdomain) (31; 32).

The S-domain recognizes and binds to the pre-tRNA substrate through the highly conserved T-psi loop of pre-tRNAs (33).

Despite the invariant structure of the pre-tRNA substrate, the RNA components of type A and type B P-RNAs show characteristic differences in their secondary structure.

The structure outside of the conserved structural core of P-RNAs is quite variable, as in group I intron RNAs (33).

Conserved regions of the RNA have evolved by concerted substitution of alternative structures, rather than by insertion and deletion of helical elements that occur in the more variable regions of the RNA.

Most of the unusual structural elements of type B PRNAs of the Firmicutes have evolved independently in Thermomicrobium roseum, a member of the green non-sulfur Bacteria (34), possibly illustrating a case of convergent evolution (horizontal gene transfer can never be excluded, however).

Interestingly, this phylogenetic dichotomy, which places Firmicutes in an original clade, is not paralleled by a particular feature of their tRNAs: the enzyme which modifies the uracil located in the T-psi loop of the tRNA to thymine, trmA, coding for S-adenosyl-L-methioninedependent tRNA (uracil-54,C5)-MTase in most bacterial clades, is mutually exclusive with trmFO, coding for an enzyme using tetrahydrofolate instead.

While TrmFO exists in most Firmicutes, it is also present in all alpha- and delta-Proteobacteria (except Rickettsiales in which the trmFO gene is missing), Deinococci, Cyanobacteria, Fusobacteria, Thermotogales, Acidobacteria, and in one Actinobacterium sp. (35).

When P-RNA sequence data from representatives of all bacterial clades was used (RNase P database http://www.mbio.ncsu.edu/RNaseP/home.html) to generate phylogenetic trees, the analysis repeatedly resulted in unstable tree topologies.

This is not unexpected because of limited length and high degree of P-RNA variability, resulting in the introduction of gaps in the alignments.

This limits the usefulness of the sequence comparison to the construction of phylogenies of narrow clades (32; 36).

For example, P-RNA is conserved in plastids and it produces a stable phylogeny for Cyanobacteria, comparable to that of 16S rRNA (37).

In the same way, P-RNA supports a phylogenetically consistent tree within the Chlamydiae (38).

In this context, it is most interesting to study bacterial clades that are outliers in the bacterial tree of life.

In the case of Aquificales, this gave an interesting result: phylogenetic trees based on structure-based sequence alignment using the program fastDNAml did not corroborate an early origin for the Aquificales P-RNAs (36).

With manual improvement of the alignments, the type A P-RNA trees grouped the Aquificales with the green sulfur bacteria, Cyanobacteria, and delta/epsilon-Proteobacteria, in general agreement with several protein-based phylogenies, but not with the usual 16S rRNA trees (39).

The major discriminating feature is the lack of helix P18 in the Persephonella marina and Sulfurihydrogenibium azorense P-RNAs.

This feature, shared with Archaea and Eukaryotes, thus seems to represent not an ancient, but a convergent trait (40).

Another group is particularly interesting to explore.

Planctomycetes form a distinct clade of Bacteria.

They display a number of unusual phenotypic features, including peptidoglycan-less cell walls, short 5S rRNA and an unlinked rrn operon organization (41).

They have a compartmentalized cell structure, unique among Bacteria.

In the PRNA of Gemmata sp.

isolates, an insert in helix P13, not found in any other member of the Bacteria, permits the construction of a tree that is consistent with the 16S rRNA tree (42).

In conclusion, the type A P-RNA tree appears to follow the standard 16S rRNA tree except for the Aquificales.

The tree position of the Aquificales has been challenged recently, however, and it is now more consistent with that generated by P-RNA (39).

It seems likely, however, that controversies will persist, as it is extremely difficult to establish robust phylogenies spanning the whole tree of life based on nucleic acids.

Even ribosomal RNA itself is prone to vary, sometimes with significant differences within a same strain (43; 44).

Nevertheless, a highly specific pattern is observed with the Firmicutes, which have type B P-RNAs, suggesting a particular pattern of evolution of this particular class of catalytic activities.

The protein component of active RNase P is encoded by rnpA gene.

In general, this gene is located near the origin of replication (when it exists) in a phylogenetically conserved way and it belongs to the group of genes used to characterize the origin (45).

As stated above, A. aeoliqus is a remarkable exception: all genes in the region are conserved, except that rnpA is missing (40).

An RNase P protein knockdown strain provided direct proof that the rnpA gene is essential in B. subtilis and, by inference, in other bacteria (27).

The strain conditionally expressing rnpA was used to screen for functional conservation among bacterial RnpA proteins from a representative spectrum of bacterial clades showing conserved function of RnpA proteins despite low sequence conservation.

Even rnpA genes from psychrophilic and thermophilic bacteria rescued growth of B. subtilis rnpA mutants (30), despite a large difference in their P RNAs.

A deletion analysis of B. subtilis RnpA defined the structural elements essential for bacterial RNase P protein function in vivo.

Despite a low degree of sequence similarity the overall fold of the protein is conserved.

In B. subtilis RnpA, a loop containing a cluster of acidic residues is thought to mediate RNA contacts via co-ordinated metal ions.

Terminal extensions and insertions in this loop remained compatible with the RnpA function in B. subtilis.

Some protein variants exhibit striking sequence extensions of unknown function, which may correlate with functional differences.

Overall, the similar three-dimensional structures of protein subunits from unrelated bacteria with B-type (B. subtilis, Staphylococcus aureus) and A-type (Thermotoga maritima) P-RNAs suggested that structural and functional conservation may be a hallmark of bacterial RnpA proteins (46-48).

Interestingly, the corresponding phylogenetic tree of RnpA matches that of P-RNA, with Firmicutes being placed out of their commonly admitted place in the standard 16S phylogeny.

This further indicates that this member of the paleome has followed a distinct evolutionary path.

This feature will be further discussed below.

### Energy-Dependent Degradation of RNA

Genes of the bacterial paleome can be divided into two more or less equal classes depending on whether or not they are essential to bacterial growth under laboratory conditions (FIG. 1B).

Essential genes, well-identified in E. coli (49) and B. subtilis (50), cannot be inactivated without loss of viability.

In contrast, a second category of paleome genes can be inactivated when cells are grown in the laboratory.

These genes correspond to a functional category of genes that are involved in maintenance and adaptation to rapid transitions under usual living conditions (12).

Degradation of weathered compounds is an essential step in the perpetuation of life.

The enzymes responsible for this function can be assumed to have played an essential role in perpetuating life over the generations, by permitting the cell to use aged structures to generate young cells with correct products.

If degradation were to be performed randomly, i.e. without screening between functional and non functional compounds (51), it is unlikely that this would be successful, given the limited number of molecules present in the small volume of a bacterium.

Hence, the production of progeny requires a selective step in the choice of the molecules that need to be conserved, in particular those absolutely required for life.

To be effective in eliminating deleterious molecules, while conserving those that are functional (even if slightly altered), this step should consume energy (52).

In this context, it is important to analyze in detail the evolution of genes encoding proteins that salvage energy.

One evolutionarily conserved component of the general RNA degradation machinery that is remarkably poised to perform this essential energy-dependent function is the degradosome, which exists in the majority of Bacteria (53).

A similar RNA degradation complex also exists in the Archaea and Eukarya, named the exosome (54).

Interestingly, compartmentalization may play a major role in the organization of these structures.

While the biochemical definition of the exosome shows that it has a simpler, less-evolved structure that cannot be split into active subcomponents, the E. coli degradosome is a protein complex with several core components that also play a role in the organization of the bacterial cell: polynucleotide phosphorylase (PNPase), ribonuclease E (RNase E), RhlB (an ATP-dependent helicase) and enolase (53; 55).

As a case in point, suggesting association of hydrolytic enzymes to these structures, RNase II (Rrp44) has been found to be associated with the exosome in Eukarya (56).

An association of the exosome with the cytoskeleton may be absent from Eukarya, as enolase (see below) is associated with the RNase E backbone in Bacteria (discussed in (57)), while in higher organisms enolase isoforms interact directly with microtubules and the centrosome (58-60), making it a core component of a cell's organization which differs considerably in Bacteria and in Eukarya.

Several other proteins associate loosely with the degradosome: polyphosphate kinase (55; 61; 62), poly(A) polymerase (63) and ribosomal protein S1 (64; 65).

We will discuss these components in the sections that follow.

It should be noted at this point that biochemical definitions of cell structures should be qualified, since even mild biochemical techniques tend to be extremely disruptive.

As a matter of fact, it is very difficult to distinguish between a “contaminant” of a purified complex and a protein that may be transiently associated with it at some point in the cell life cycle.

In this respect, it may be worth noting that the association of ribosomal protein S1 to the degradosome was predicted in silico well before this was demonstrated biochemically (64; 65).

Furthermore, while systematically practised by biochemists all over the world, breaking open a cell in the presence of dioxygen is extremely disruptive by nature.

Any complex where a ferrous ion is not extremely tightly bound to its partner will simply vanish, whereas in silico analyses suggest that iron may play a much more important role than previously suspected (13), well in line with scenarios about the origin of life (8; 10; 11; 13).

In this context, it may be relevant to take into account in silico studies that suggest that some objects are synthesized in the same zone in the cell and may therefore tend to interact (66; 67).

### Phosphorylases

Degradation of polynucleotides by phosphorolysis is ubiquitous.

Remarkably, phosphate is not only part of the backbone of nucleic acids and a variety of other building blocks of structural components of the cell (membranes, teichoic acids and the like), but it is also a major player in the management of energy (68).

In this respect, it is worth noting that RNA degradation can proceed via the formation of energy-rich bonds: the energy present in phosphodiester bonds is recovered as the RNA molecules are degraded, by using phosphate for phosphorolysis instead of the ubiquitous water-mediated hydrolysis.

Degradation by phosphorolysis produces nucleoside diphosphates that play a central role in the channelling of the cell's metabolism towards anabolism.

Energy saving is of major importance when cells enter into stationary phase of growth, where many components of the cell age with a concomitant loss of energy synthesizing capacity, while the cell needs to produce a rejuvenated progeny when growth resumes.

We have proposed that this requires a selective process which uses energy to discriminate between newly synthesized components and old ones ((69) and see below).

The salvaging of energy by phosphorolysis of RNA would be admirably suited to this purpose, especially if polyphosphates, the product of another loosely associated component of the degradosome, could be used to restore the triphosphate levels of ATP and GTP when the cell reconstitutes a functional ATP synthase and/or glycolytic flux.

It seems therefore quite remarkable that polyphosphate kinase is indeed associated with the degradosome.

A second property of nucleoside diphosphates is that they are almost ubiquitously preferred over ribonucleoside triphosphates for the biosynthesis of deoxyribonucleotides (there are only a few exceptions where NTPs can be used (70), but this is a backup of the normal NDP route).

Surprisingly, the role of RNA phosphorolysis in generating the NDP precursors required for DNA synthesis, which might play a role in optimisation of the relative distribution of DNA nucleotides to RNA, (as remarked by Seymour Cohen as early as 1960 (71)), has only rarely been pointed out, despite its importance (64).

Briefly, while the pyrimidine biosynthesis pathway goes through UDP (which, interestingly, should not enter DNA), CDP is not formed directly, creating a relative starvation for this precursor of dCDP, and consequently dCTP.

This creates a general tendency for most genomes to increase their A+T content (72).

The consequence of this organization of metabolism is that incorporation of cytosine into DNA results essentially from processes where CTP is recycled to CMP, unless CDP can be produced directly, which is exactly the outcome of RNA phosphorolysis (66).

Ribonuclease PH and PNPase, which descend from a common ancestor, are the two avatars of these ubiquitous phosphorolytic enzymes.

PNPase is a structurally complex enzyme (73-76), that is certainly the product of a very intricate evolution scenario.

It comprises five domains: two RPH-like domains (PNP1 and PNP2), one alpha helical domain, and two RNA binding domains (KH and S1 domains).

We will discuss RNase PH before considering the evolution of PNPase, since the latter is organized around RPH domains.

Except for the Mollicutes, Bacteria generally possess one or other of these enzymes, or both (3).

Ribonuclease PH RNase PH (pre-tRNA phosphorylase, mistakenly referred to as tRNA nucleotidyltransferase in several UniProtKB (77) entries, probably because the reaction is reversible in vitro (78)) was discovered as an important enzyme involved in the maturation of the 3′-ends of tRNAs (79).

The enzyme has a ring structure made up of six subunits, arranged as a trimer of dimers (80; 81).

This structure allows the binding to tRNA precursors and the trimming of their 3′ extremities (80; 82; 83).

The CCA motif is then added to these extremities by tRNA nucleotidyltransferase (Cca), one nucleotide at a time (84).

The RNase PH ring structure appears to be the model from which PNPase and the eucaryotic exosome have evolved, by domain duplication and fusion with a variety of other domains (85).

Its strong sequence conservation in distantly related organisms argues in favour of a very constrained activity.

Its function appears to sometimes overlap with that of PNPase, since some organisms have only one or other of these enzymes (3; 12).

RNase PH also forms the core structure of the archaeal exosome.

Polynucleotide phosphorylase PNPase is also a trimeric complex, forming a doughnut-shaped structure where the RNA-binding domains create a central pore where RNA enters.

Each subunit contains five domains, including two RNase PH domains (PNP1 and PNP2), one alpha helical domain, one KH domain, and one S1 domain (76).

Phylogenetic analysis suggests that PNPase was formed via a duplication of an ancestor of RNase PH.

The current distribution of these domains on the tree of life suggests that the PNP domain predated the separation between Archaea, Eukarya, and Bacteria.

PNP2 and RNase PH are more closely related to each other than either one is to PNP1, suggesting a functional specialization of PNP1 during evolution.

The function of PNP1 is unclear, but it appears to synthesize guanosine 3′-diphosphate 5′-triphosphate (pppGpp) in Streptomyces antibioticus (86), while the phosphorolytic catalytic site is thought to be located within the PNP2 domain in both this organism and in E. coli PNPase.

The pppGpp-related function should probably be explored further, as it provides a functional link with another component of the degradosome, polyphosphate kinase, and (p)ppGpp has been shown to control polyphosphate metabolism (87).

PNPase belongs to the degradosome in Proteobacteria, where it is associated with RNase E, the scaffold for the different components of this RNA degradation complex (53).

It is also weakly associated with a variety of other enzymes such as Poly(A) polymerase, involved in non-templated RNA synthesis, and CspE, an RNA binding factor that recognizes the sequence AAAUUU (65).

Finally, the conserved codon usage bias of degradosome components suggests that other components, including other hydrolytic RNases, may be synthesized in the same region of the cell and be loosely associated with it (66; 67).

The situation is different in Firmicutes, where there are generally few orthologs of RNase E, but this does not preclude the existence of a functional degradosome-like complex (see below).

Indeed, it is remarkable that PNPase co-evolves with enolase and the central enzymes of glycolysis in the Firmicutes, as it does in the Proteobacteria, suggesting that at least part of the degradosome may be conserved throughout evolution of Bacteria (Table I).

A domain analysis of chloroplast PNPase revealed discrete functions in RNA degradation, polyadenylation, and sequence homology with exosome proteins (74).

The evolution of PNPase and related proteins is therefore inseparable from the evolution of its various domains.

A parallel situation is encountered with RNase E (see below).

The S1 domain The S1 domain is typical of an ancestral RNA-binding domain.

It gets its name from ribosomal protein S1 (RpsA in the gamma-Proteobacteria), which contains six similar sub-domains (S1 domains) and which interacts with the 30S subunit of the ribosome (88).

RpsA is essential for ribosome binding site (RBS) recognition in these bacteria.

In contrast, its homologue in Firmicutes, YpfD, while comprising four S1 domains, does not appear to bind to the upstream region of messenger RNAs to permit initiation of translation.

The lack of this function explains why RBSs are highly conserved in these organisms, with the consequence that Firmicute genes are usually highly expressed in Proteobacteria (and often toxic for this reason (89)), making them better hosts for genetic engineering than Firmicutes.

Interestingly, the S1 protein of E. coli recognizes a pseudoknot in tmRNA (90), and YpfD has a similar function in vitro (91).

This suggests an interesting role for this presumably primitive domain in the recognition or presentation RNA.

The S1 domain is found in several other RNases beside PNPase, notably RNase II (92) and RNase E (93).

RpsA is also required for the specificity of ribonuclease RegB from bacteriophage T4.

It recognizes the core motif of the RBS, GGAG, acting as a presenting protein to the endonuclease (94; 95).

This may account for the function of YpfD in Firmicutes, which could also play the role of presenting RNA to their still elusive RNA degradation apparatuses (66; 91).

In summary, the S1 domain appears to be an RNA-presenting domain, that has been recruited by several enzymes by acquisitive evolution.

Ribosomal protein S1 in delta-Proteobacteria is particularly long, and coded in a different gene environment compared to other organisms.

It also evolves along a similar tree to other proteins which appear to be specific to this clade (96).

All these features suggest that the S1 domain is associated with sequence- or structure-specific RNA motifs that define specific properties of bacterial clades.

The KH domain The K homology (KH) domain is also an ancestral and widespread RNA-binding domain.

It has been detected by sequence similarity searches in eukaryotic proteins, such as heterogeneous nuclear ribonucleoprotein K and ribosomal protein S3 (97).

This motif is present in the EngA (YphC) GTPase of B. subtilis and T. maritima, involved in ribosomal 50S subunit assembly.

It is present both in Era, a small GTP-binding protein essential for cell growth in E. coli, and RbfA, responsible for a step of ribosomal RNA maturation (98).

It is also present in the RsmA (CsrA) protein which interacts with small regulatory RNAs (srRNAs, often named with the misleading <<non-coding>> tag, sncRNAs) in Proteobacteria, presumably providing specificity to the recognition of RNA substrates (99).

It has also been proposed that the KH domain of PNPase plays a role in substrate recognition (100) and this may account for the fact that PNPase is a key player in degradation of srRNAs (101).

Its pattern of evolution makes its function less clear than that of the S1 domain.

This may be related to the observation that the proteins with a KH motif provide a rare example of protein domains that share significant sequence similarity in the motif regions, but possess globally distinct structures (97).

### ATP-Dependent RNA Helicases

Some studies have shown that PNPase has a beta subunit in addition to its catalytic core.

It was initially thought that this subunit was the enzyme enolase, but further work showed that it is in fact the ATP-dependent RNA helicase RhlB (102).

More generally, ATP-dependent helicases are conserved components of the degradosome (103).

Counterparts are also present in the exosomes of Eukarya, where an energy-dependent process is important for mRNA surveillance (104).

The RNA helicases might be important for substrate recognition and recruitment, and for the activation of the catalytic activities of various types of degradosomes or exosomes, sometimes as isolated complexes (105).

There are a considerable number of DEA[D/H]-box ATP-dependent RNA helicases involved in RNA folding, maintenance and degradation in Bacteria.

This is the case for the ribosome-associated cold-shock protein CsdA in E. coli, and CshA (YdbR) and CshB (YqfR) in B. subtilis.

Interestingly, another putative DEAH-box ATPdependent RNA helicase, HrpA, is involved in the processing of a fimbrial mRNA molecule in E. coli (106).

Homologs of this protein are widely present in bacterial clades (but seemingly absent from the Firmicutes) and it appears to co-evolve with RNase E.

This indicates that when investigating the evolution of ribonuclease activities, we should also explore in depth the many genes of unknown function that code for proteins having features related to the DEA(D/H)-box ATP-dependent helicases and which may in fact be involved in specific RNA processing.

Because their activity depends on ATP hydrolysis, it is tempting to speculate that their role is similar to that of EF-Tu in translation, using energy to discriminate between substrates and degrade only those that are relevant via a kinetic proofreading mechanism (52; 107).

Indeed it appears that the ATPase activity of RhlB is regulated allosterically (108) and that one role of the enzyme is to control RNA misfolding (109).

Like other energy-dependent helicases, it could also be involved in the processes of surveillance and degradation of damaged RNA molecules.

Targets might include stable RNAs and RNAs modified by a variety of chemical processes related to reactive oxygen species, errors in transcription or other modifications of RNA, often found in Eukarya, but poorly documented in Bacteria.

Bacteria growing in cold or very cold conditions should be explored in priority, as it can be expected that RNA folding and stability at low temperature might interfere with smooth regulation (110).

As this would require a full study in itself, this area falls outside of the scope of this review.

Evolution of the Association of Energy with RNA Degradation: Polyphosphate Synthase/Kinase and Enolase

Poly(P), a ubiquitous source of energy, generally assumed to play the role of phosphate storage, is systematically associated with RNA degradation and in particular to the degradosome (62).

Poly(P) has been detected in various amounts in all organisms tested to date (111).

The pathways of its metabolism are not understood in detail, however, despite the extensive work of the late A.

Kornberg and his co-workers, who demonstrated the remarkable features of poly(P)-deficient strains (112).

Three biosynthetic pathways are known, but they apparently fail to account for the bulk of poly(P) accumulation.

The major enzyme of poly(P) metabolism, polyphosphate kinase (PPK), processively and reversibly catalyzes phospho-transfer between ATP and poly(P).

McMahon and coworkers, who analyzed the metagenome of sludge plants, determined a general tree of PPK evolution in the corresponding genome samples (113).

Mycobacteria and Streptomycetes are present, as well as some Firmicutes (but B. subtilis and its neighbours, as well as Streptococci are absent).

One also finds there some Cyanobacteria as well as Alpha-Proteobacteria.

Another class of processive polyphosphate kinases, PPK2, prefers GTP and is widely conserved in Bacteria (114).

Many organisms have both PPK and PPK2 in their genomes (D. radiodurans and gamma-Proteobacteria such as P. aeruginosa, Vibrio cholera . . . ), a few have only PPK2, some only PPK (Enterobacteria, the Bacillus cereus complex, Staphylococcus epidermidis and S. haemolyticus, but not S. aureus . . . ), and many have neither PPK nor PPK2 (B. subtilis, Sulfolobus solfataricus, Thermotoga maritima . . . ).

The latter, however, have enzymes, namely PpnKA (YjbN) and PpnKB (YtdI), involved in the poly(P)-dependent biosynthesis of NADP (this activity has been biochemically established in Mycobacterium tuberculosis (115)) and might account for the missing (or a subunit of the missing) polyphosphate kinase.

In B. subtilis ppnKA is essential and is in synteny with several genes involved in RNA metabolism or related activities.

Interestingly PpnKA co-evolves with several RNase-related genes in Firmicutes, including RnmV, MrnC(YazC), YacP, YhaM, RnjB(YmfA), CshB(YqfR), RnhC(YsgB), NrnA(YtqI), YusF, YybT (Table I).

Considerable work is needed to explore the link between poly(P) and RNA degradation further.

It is clear, however, at this point that there is a strong functional association between this source of energy and some ribonuclease activities.

The second energy-managing component of the degradosome is enolase (53).

Besides its structural role in the bacterial cytoskeleton via the RNase E scaffold ((116), see also (57)) the presence of enolase in the degradosome complex makes sense since, by providing phosphoenolpyruvate to nucleoside diphosphokinase (NDK), it permits the regeneration of GTP from the GDP produced by PNPase (64; 117).

NDK exists in two forms in E. coli, in Pseudomonas aeruginosa, and in the Mycobacteria.

The long cytoplasmic form of NDK binds to succinyl-CoA synthetase and exhibits a low substrate specificity, whereas the short membrane-bound form is strongly and specifically associated with pyruvate kinase, and synthesizes GTP from GDP and phosphoenolpyruvate (for a review see (118)).

Enolase follows a complex phylogenetic pattern: while clearly separating Archaea and Eukarya from Bacteria, the fine distribution in bacterial clades looks somewhat erratic (119), as does that of various other components of the degradosome.

### Poly(A) Polymerases

Two processes require nucleotide addition at the 3′ end of RNAs, tRNA nucleotidyl transfer (which adds CCA, as already discussed above), and the addition of poly(A) stretches, which modifies a variety of RNAs and is involved in RNA turnover.

tRNA nucleotidyl transferase is widely distributed.

Interestingly its distribution appears to parallel that of a small RNA binding protein, Hfq, which is also widely present in Bacteria (for that matter, it is present in most Firmicutes, with the exception, perhaps, of the Streptococci).

In this respect, an ancestral function of this protein might be interaction with tRNA and tRNA nucleotidyl transferase, more so than its role in increasing the processivity of poly(A) polymerase (120).

Many features of mRNA decay differ between the Bacteria and the Eukarya.

Polyadenylation plays a role in the decay of some bacterial mRNAs, as well as in the quality control of stable RNA.

However, while poly(A) polymerase is widely conserved, polyadenylation of mRNAs appears to serve a very different function in prokaryotes than its primary role in eukaryotes (121).

In E. coli, the main polyadenylating enzyme is poly(A) polymerase I (PAP I), but the addition of 3′ tails also occurs in the absence of PAP I via the synthetic activity of PNPase when the ADP levels are high enough, indicating a functional interaction with adenylate kinase.

In S. coelicolor, PNPase is likely to be responsible for 3′-end poly(A) addition.

In the same way, in Synechocystis and spinach chloroplast, 3′-end poly(A) addition is carried out by PNPase.

B. subtilis lacks an identifiable PAP I homologue.

However, analysis of 3′-tails revealed a similar pattern in wild-type and PNPase-deficient strains, indicating the existence of an alternative poly(A) polymerase activity in this organism (122).

We have already stressed that B. subtilis and many Firmicutes possess an RNA degradation system that differs significantly from that of most Bacteria, with genes which tend to co-evolve.

It will therefore be of interest to explore whether one of the members of this set of genes of unknown function displays poly(A) synthesis activity (Table I).

### Hydrolytic Nucleases

### Hydrolytic Endonucleases

The chemical activity of water is extremely high in all biological systems.

Therefore, the catabolism of macromolecules, nucleic acids in particular, employs hydrolysis reactions ubiquitously.

While phosphorolysis salvages energy, hydrolysis is not energy saving, and as such it can only discriminate its substrates by passive selection (52), serving as a general scavenging activity, sometimes with associated regulatory properties.

It is therefore not unexpected that the degradosome and the exosome have hydrolytic RNase activities (such as RNase E and probably others in the case of the degradosome) to complement the phosphorolytic activity of PNPase (66).

The three major classes of RNases use polynucleotide hydrolysis for RNA degradation: those responsible or endonucleolytic attack, processive degradation from the 3′-end and degradation from the 5′-end.

The first process usually takes into account sequence and/or structural properties of the RNA substrate, and, as such, it may be associated with helicases and exonucleases.

Exactly as in the case of PNPase and RNase PH, there exists a widespread core endonuclease framework, descending from a common ancestor, which has been characterized in both RNase E and RNase G.

The RNase E/G family preferentially cleaves A/U-rich single-stranded regions of otherwise structured RNAs.

Interestingly, as stated in the cautionary word at the beginning of this review, when this family is absent and replaced by members of the RNase J1/J2 family (see below), the AU-rich single stranded cleavage function is conserved; i.e. this function is conserved even if the enzyme structure is not RNase E/G members play an essential role in RNA maturation and decay.

Our current understanding of these enzymes originates from studies of E. coli RNase G and RNase E, as well as studies in some isolated examples of particular species.

Ribonuclease G RNase G is a remarkable endonuclease, in that it senses the monophosphate located at the 5′-end of the RNA to be cleaved (123).

It is involved in the maturation of pre-16S rRNA and its evolution parallels that of 16S rRNA, suggesting that it is related to an ancestral form that gave rise to present day RNase G and E.

RNase G is also involved in the degradation of the mRNA coding for enolase in E. coli, creating an interesting RNA degradation feedback loop (124).

RNase G was previously named CafA, because its overproduction led to an alteration of the cytoskeleton through formation of an axial filament, suggesting that the protein is, functionally at least, associated with structuration of the cytoplasm (125; 126).

It may therefore interfere with the organisation of the degradosome, possibly because of an interference with the RNase E scaffold, via some type of interaction with a component associated to the degradosome (66).

The core catalytic center of RNase G is highly similar to that of RNase E, so that both proteins are often classified within a single category, RNase E/G (3), as in A. aeolicus (127), but RNase G cannot fully complement an RNase E defect in E. coli, except with highly overproduced variants (128).

It must be noted, however, that the number of cases where the actual function of the enzyme has been assayed is very limited.

In one case where it has been studied, it was shown that the RNase E/G homologue (MycRne) from M. tuberculosis is a 5′-end-dependent endoribonuclease with some overlap with RNase E function, such as 5S ribosomal RNA processing (129).

Since Mycobacteria also have RNase J1 (Rv2752c), an enzyme that processes 16S rRNA in B. subtilis (130), it will be interesting to see which enzyme catalyzes 16S rRNA maturation in this organism.

Sequence analysis of the distribution of RNase G in genomes is somewhat inconsistent, suggesting that there has been either convergent evolution for its function, or that a variety of other proteins underwent acquisitive evolution of the corresponding function (see below).

While this enzyme domain is frequently observed in Bacteria, it does not apparently have many counterparts in A+T-rich Firmicutes (but it has in some (3)), where ribosomal RNA maturation is, potentially at least, performed by structurally different enzymes (see below).

Ribonuclease E In E. coli, RNase E is considered the major endoribonuclease responsible for the degradation and processing of mRNAs and srRNAs (131), as well as stable RNAs, in particular tRNA maturation (132).

It is composed of three domains, an N-terminal catalytic region comprising an S1 domain (93), a central RNA-binding domain, and a Cterminal scaffold region responsible for binding of the associated proteins (53).

Like RNase G, RNase E cleaves RNA internally, while its catalytic power may be determined by the 5′ terminus of the substrate, even if this lies at a distance from the cutting site ((133), but see (123)).

RNase E is present in most Bacteria, with the same core structure, but with a variety of extensions at the N- or C-terminus, sometimes both.

It is absent however in many Firmicutes and in Mollicutes.

Long extensions of RNase E permit it to become a component of the bacterial cytoskeleton (116).

The sequence of the C-terminal half of RNase E is not highly conserved evolutionarily, suggesting that there may be some diversity among RNase E interactions with other components in different organisms.

Furthermore, there could even be epigenetic variation in the polypeptide sequence due to mRNA sliding in the ribosome at a specific AGCU site (ribosomal hopping) as a function of the environment (57).

As an example, the Synechocystis sp. RNase E homologue does not permit assembly of E. coli degradosome components (134).

In Streptomyces coelicolor, RNase ES is a structurally shuffled RNase E homologue showing evolutionary conservation of functional RNase Elike enzymatic activity.

This suggests the existence of degradosome-like complexes in at least some Gram-positive bacteria (75).

As already indicated, there is no conserved counterpart of RNase E in B. subtilis and many Firmicutes (see section on the RnjA/B family below).

In E. coli, the activity of RNase E is regulated by several effectors, RraA (135) and RraB (136) in particular.

These proteins appear to alter the composition of the degradosome and it is therefore interesting to analyse their corresponding phylogenies.

Overall, it appears that the trio of RNase E, RraA and RraB co-evolves, and this could probably be used to infer the function of a protein belonging to the RNase E/G class in the absence of experimental data.

There are a few instances where RraA and RraB seem to be both present, while an obvious RNase E/G counterpart is missing: Arcobacter butzleri, for example, representing the epsilon-Proteobacteria, and the representatives of the Thermales/Deinococcales, Deinococcus radiodurans and T. thermophilus.

In the Firmicute Geobacillus kaustophilus, the situation is similar, while in Bacillus pumilus one observes a fairly divergent pair of these proteins.

They may represent relics of an RNase E-type degradosome that was present in Firmicutes (it can be observed in Bacillus cereus and partially in Listeria innocua) and subsequently displaced by a totally divergent degradosome structure (see discussion) that became a specific marker of the Firmicute genome organization.

Alternatively, but perhaps less likely, they could represent attempts by the Gram-negative degradosome to invade an older degradosome structure present in the ancestors of the Firmicutes, by horizontal gene transfer with subsequent loss of the functionally duplicated elements.

Finally, in Rubrobacter xylanophilus, there is a weak conservation of these regulators, with no 21 obvious Rne-related sequence.

Because the whole system is present in the other Actinobacteria, this could simply reflect a fast evolution of the structure in Rubrobacteridae.

It should also be remembered that genome sequences are not 100% accurate, so that experiments would be needed to make a strong point from the apparent absence of this gene.

Ribonuclease M5 RNase M5 (RnmV, YabF) is a highly specialized endonuclease that appears to be specifically involved in maturation of 5S RNA in most Firmicutes (including Mollicutes) and Fusobacteria.

It is absent elsewhere, except in the Spirochete Borrelia burgdorferi (3).

This RNase belongs to the core class of proteins that co-evolve in Firmicutes, consisting of RnmV, MrnC (YazC), YacP, YhaM, RnjB(YmfA), CshB(yqfR), RnhC(YsgB), NrnA(YtqI), YusF and YybT, and which seems to be a hallmark of this particular class of organisms (Table I).

Metallo-□-lactamases The metallo-□-lactamase fold, known for its important role in resistance to penicillinrelated antibiotics, is widely spread in bacterial proteomes.

The corresponding protein fold is used in a large variety of hydrolytic reactions (137).

In particular these proteins are often found as nucleases, acting either on DNA or on RNA (138).

Specificity appears to come from a variety of domains associated to the hydrolytic alphabetabetaalpha-fold and its conserved catalytic residues.

Two major families, without a very consistent phylogenetic distribution, play an important role in RNA degradation.

Rnz RNase Z (ElaC, Trz, Rnz, YqjK in B. subtilis) is an endonuclease belonging to the metallo-beta-lactamase family (with a core HXHXDH motif).

It is generally involved in tRNA maturation (139) and has been shown to process CCA-less tRNA precursors in B. subtilis (140).

It is found in Firmicutes (not in Mollicutes), Cyanobacteria, Spirochetes and is fairly widespread elsewhere, including in some gamma-Proteobacteria.

It is identical with RNase BN in E. coli, where all tRNA molecules are coded by a CCA containing genes, making the function of this enzyme fairly enigmatic (141).

It has been shown to play a significant role in mRNA decay, in conjunction with RNase E (142).

RNase Z displays less identity, but is conserved over its whole length in Thermotogales (139; 143).

In general, its distribution is not consistent with phylogenetic trees of proteins with related functions, suggesting widespread horizontal gene transfer (139).

RnjA/B Interestingly, RNase Z shows some sequence similarity with a large class of RNases, RNase J1 (RnjA) and J2 (RnjB), which play the role of RNase E where it is absent, in particular in B. subtilis (144).

RnjA (YkqC) has been found to be involved in 16S rRNA maturation (130).

Remarkably, both RnjA and RnjB have an additional activity, that of a long sought after 5′-3′ exonuclease (145).

Both activities have been demonstrated in T. thermophilus (146).

Most Firmicutes contain both enzymes.

However, as for RNase E and G, their activities appear to be overlapping and it would be extremely interesting to perform a detailed study of the presence of the enzyme in one or several copies in different organisms.

Homologues of RnjA/B are present also in Actinobacteria, Cholorflexi, Deinococcus/Thermus, Fibrobacteres/Acidobacteria, Cyanobacteria, and, remarkably, in some alpha- and epsilon-Proteobacteria.

It is also present in a few gamma-Proteobacteria, and in several delta-Proteobacteria.

Others Metallo-beta-lactamase superfamily proteins with related sequences are widespread, but their function is often unidentified: the putative B. subtilis metallo-dependent hydrolases YflN, YybB, YycJ, YqgX display some sequence similarity with the Rnz/RnjA/B class of RNases and tend to co-evolve with the RnjA/B proteins.

It will be interesting to investigate their possible role in specific RNA metabolism.

Indeed mRNA turnover might be modulated by different RNases, providing an additional control to gene expression.

Ribonuclease III and ribonuclease MrnC RNase III (Rnc, RanA, AbsB) is a ubiquitous endonuclease that participates in the maturation of ribosomal RNA from precursors.

The protein belongs to the paleome, and it evolves following a tree that parallels that of the 16S rRNA.

The structural fold of the protein is present in Eukarya as well, where proteins of this class have a major role in RNA-regulated processes.

Its phylogeny in individual species and in the alpha-Proteobacteria has been explored in some detail (147; 148).

There are many situations when mRNA is cleaved during the translation process, in particular when ribosomes are stalled by amino acid starvation or other physico-chemical transitions (such as rapid temperature up or downshift).

It has been shown that, under such conditions mRNA molecules are often cleaved by ribonucleases (149) and that tmRNA is involved in rapid degradation of the truncated RNAs.

A major role of the tmRNA is to extend translation of truncated proteins in statu nascendi, with a peptide tag that send them to the ClpXP degradation system (150).

Furthermore, the tmRNA system facilitates the release of the truncated mRNA from the stalled ribosome and allows its rapid degradation by RNase R (see below) to prevent production of aberrant polypeptides (149; 151).

The proteins responsible for cleavage of the stalled mRNAs have not yet been identified, but they are probably endonucleases associated with the ribosome (152).

The related protein MrnC (YazC), with a typical RNase III fold is involved in 23S RNA maturation in B. subtilis (153).

It is present in Firmicutes, (not Mollicutes) and present in Cyanobacteria and Thermotogales, but with a fairly divergent sequence.

It belongs to the group of co-evolving RNases present in Firmicutes and highly specific to this bacterial family (Table I).

Miscellanea The YjgH protein is a putative L-PSP (mRNA) endoribonuclease (PFAM PF01042) that is similar to ribonuclease UK114 from mouse (154) and is possibly responsible for the inhibition of translation by cleaving mRNA in E. coli.

These RNases cleave phosphodiester bonds only in single-stranded RNA.

YjgH co-evolves with YjgI a putative oxido-reductase that may act on nucleotides.

Enzymes involved in the degradation of RNA molecules containing modified nucleotides have not yet been explored.

Curiously, YjgH has a fold (PDB entry 1 PF5) that is similar to that of the YjgF/TdcF family of proteins, and that have been crystallized, but which do not yet have a well-identified function.

The putative catalytic centre residues are not conserved, however.

An inference of functional from the sequence alone may be extremely misleading.

The most likely counterparts of the protein can be found in alpha-Proteobacteria.

YmdA is an essential putative phosphohydrolase that is conserved among Firmicutes (155).

It contains a HD/KH domain strongly suggestive of RNA binding (156).

Its activity should therefore be explored as a priority, especially as it belongs to the group of proteins co-evolving with other RNases in Firmicutes.

Interestingly, it is also conserved in Planctomycetes, in delta/epsilon-Proteobacteria and in Thermotogales.

In the same vein, YusF, which contains the Toprim domain found in RNase M5, belongs to the coevolving family of RNases associated with Firmicute.

It is also possible, however, that this gene product is more functionally related to the DNA primases (156).

The B. subtilis YfkH protein is incorrectly annotated in many genomes as the tRNAprocessing ribonuclease BN (157)), due to a mis-identification of the gene associated with this activity (158).

RNase BN was later shown to be encoded by the elaC/rnz gene and to be identical with RNase Z (above) (141).

It should be noted that YfkH is a typical Integral Inner Membrane Protein (IIMP) (159), suggesting that its activity is membrane associated.

Counterparts of YfkH appear to be almost ubiquitous (no obvious counterpart in Thermotogales, but this may be due to specific evolution of IIMPs at high temperature), making its function the more interesting to identify, even if it is unlikely to have ribonuclease activity.

Cleavage of RNA/DNA hybrids: Ribonucleases HI HII HIII RNase H enzymes encode an essential function: they degrade the RNA-DNA hybrids formed during the priming of DNA replication, essentially on the lagging strand.

There are three types of this RNase, named RNases HI, HII and HIII.

The molecular evolution of this family of enzymes, often present in multiple copies in genomes, has been used to examine the implications of functional redundancy for gene evolution (160).

It appears that the RNase H group evolved in such a way that RNase HI and HIII are mutually exclusive despite appearing to have fairly similar substrates.

In contrast, RNase HII, which co-evolves with DnaA and PolA, as expected considering its function in degrading RNA in DNA-RNA hybrids, can co-exist with either RNase HI or HIII.

The latter, however, lacks the sequence corresponding to a basic protruding region of the E. coli R Nase HI (161).

RNase HI (RnhA) is present here and there in various clades or species, but is absent from most Firmicutes and the Thermotogales.

In contrast, RNase HIII (RnhC) belongs to the family of RNases that co-evolve in A+T-rich Firmicutes in a highly specific manner (Table I).

Finally, the protein family DUF458 (YkuK) is distantly related to RNase H (162).

In sporulating Firmicutes, the protein co-evolves with several proteins involved in sporulation/germination, and it is present in thermophilic organisms (A. aeolicus, T. tengcongensis and Thermotogales) as well as in Clostridium acetobutylicum.

It is not possible to know at this point whether this protein is involved in DNA or RNA metabolism.

### Hydrolytic Exonucleases

3′-5′ exonucleases Ribonuclease II and ribonuclease R 3′-5′ exoribonucleases that processively hydrolyze single-stranded RNAs, generating 5′ mononucleotides and liberating short oligoribonucleotides (typically less than 5 nts) as final degradation products, constitute a distinct functional class.

RNase II and RNase R of E. coli are close kins, but they are probably involved in different activities.

An analysis of the codon usage bias of the RNase II (Rnb) gene in E. coli shows that it may be synthesized in the same region of the cell and suggests that is functionally associated with the activity of the degradosome (66; 67).

Its catalytic core is surrounded by three RNA-binding domains.

There is a typical S1 domain at its C-terminus that is critical for RNA binding, suggesting some sort of screening mechanism for specific sequences.

It is highly related to RNase R (Rnr, VacB, YjeC in E. coli), which has a similar degradative capacity, but may be involved in degradation of misfolded ribosomal RNA (163).

Both enzymes appear to have overlapping activity and structural properties (the situation is somewhat similar to that of RNase E/G and RNase J1/2) (164).

Like PNPase, RNase R can degrade structured RNAs (163).

RNase R is associated to the degradosome in Pseudomonas syringae (165).

RNase II and R are so similar that, unless explicit experiments are performed to identify their activity, it is not yet possible to know whether a genome possesses either of the activities, or both, using sequence information only.

That said, RNase R is generally longer than RNase II by about 200 residues, some 100 of which or more are located at the C-terminus.

Their phylogenetic pattern parallels that of 16S rRNA.

Both enzymes work best on poly(A) in vitro (164).

However, while RNase R shortens RNA processively to di- and trinucleotides, RNase II becomes more distributive when the length of the substrate reaches approximately 10 nucleotides, and it leaves an undigested core of 3-5 nucleotides (56).

Thus, several types of short oligonucleotides (“nanoRNAs”) accumulate in the cell as the consequence of the activity of these enzymes (see below).

The activity of RNase II is modulated in E. coli by the Gmr (YciR) GGDEF diguanylate cyclase/phosphodiesterase protein (166).

As this family of proteins is very frequent, it is difficult to know at present whether this type of RNase modulation is widespread.

It should be noted, however, (see below) that the degradation of cyclic-di-GMP generates the dinucleotide pGpG, which must ultimately be cleaved by a nuclease.

Ribonuclease D and ribonuclease T RNase D (Rnd) (167) and RNase T (Rnt) (168) catalyze tRNA-end turnover.

The former is present in Actinobacteria, Aquificales, Bacteroidetes/Chlorobi, Cyanobacteria, Planctomycetes, alpha-, gamma- and some delta-Proteobacteria, and some organisms (alpha-Proteobacteria in particular) appear to have two variants, a long and a short form of the enzyme.

In contrast RNase T is unambiguously present only in the Proteobacteria (but not in delta/epsilon-Proteobacteria).

Both seem to be totally lacking in Firmicutes and in Thermotogales.

A putative exonuclease of the same family, KapD in B. subtilis, is similar to eukaryotic histone mRNA exonucleases.

It is involved in the control of the KinA pathway to sporulation (UniProtKB/TrEMBL O24685).

This could correspond to an effect on a specific RNA involved in the process.

The fate of the many srRNAs that exist in Bacteria is extremely poorly known and it is expected that ribonucleases are involved in their turnover.

However, nothing is known about the real substrates of this putative enzyme, and it is more or less randomly distributed in the bacterial phylogenetic tree (Bacilli and some Clostridiales, some Cyanobacteria, Planktomycetes, Alteromonadales, some Pseudomonas sp., Campylobacter jejuni and Thermotogales).

5′-3′ exonucleases An examination of the distribution of the 5′-3′ exonuclease PolA domain among 250 bacterial genomes showed that all Bacteria, but not Archaea, possess this domain (169).

As far as RNA degradation was concerned, however, the situation was far from clear, until it was found that RNase J1 has 5′-3′ exonuclease activity in addition to its endonuclease activity (145).

As a matter of fact, the difficulty in identifying this activity in Bacteria up to now may have come from the want of an extra step: the 5′-3′ exoribonuclease activity of RNase J1 only functions on a 5′ monophosphorylated RNAs.

Thus, while exonucleolytic RNA degradation can begin from the 5′-end, this process requires an additional step, that of a 5′-triphosphatase, releasing to produce a 5′-monophosphorylated RNA molecule.

Messenger RNA 5′ pyrophosphatase An investigation of such an activity was only recently undertaken in E. coli, with the identification of the RNA pyrophosphohydrolase RppH (YgdP, NudH), tentatively present (NUDIX hydrolases are ubiquitous) in most Proteobacteria, but not in delta-Proteobacteria, and present in Leptospira interrogans, but not in Thermotogales (170).

The phylogenetic tree pattern of this protein is not that of the 16S rRNA.

It appears to be 27 absent from all other bacterial families (but other distant NUDIX hydrolases are systematically present).

This requires experimental identification of the cognate function, which could be performed after recruitment of completely different structures.

In addition to NUDIX hydrolases, many candidates could be found among the enzymes that release phosphate or pyrophosphate and recognize nucleotides, in particular adenylyl and guanylyl cyclases, which have never been assayed for this activity, despite their large number in some species (171; 172).

Putative ribonuclease YacP Using sensitive sequence profile searches and contextual information associated with domain architectures and predicted operons, Anantharaman and Aravind identified a putative 5′-->3′ exonuclease NYN domain that shares a common protein fold with two other previously characterized groups of nucleases, namely the PIN (PilT N-terminal) and FLAP/5′-->3′ exonuclease superfamilies.

The Bacillus subtilis protein YacP, which belongs to this family has been proposed to be involved in ribosomal RNA maturation because of its chromosomal context (173).

It appears to be particular to Firmicutes and Cyanobacteria, being also present in Actinobacteria, Rubrobacter xylanophilus and Chloroflexi, Chloroflexus aurantiacus.

It belongs to the co-evolving family of proteins that contains several Firmicute-specific RNases (Table I).

### NanoRNases

At this point of our inventory it appears that we have identified all of the steps involved in RNA degradation except for one: as we have seen, processive exonucleases release nanoRNAs in the cell.

This is potentially extremely toxic, both for replication and for transcription, especially because the open replication and transcription bubbles can accommodate oligonucleotides of as many as 7 residues, with a strong effect of 5-mers (174).

NanoRNA degradation is therefore an essential function.

In E. coli short oligoribonucleotides are degraded by a processive mechanism, after attack at a free 3′ hydroxyl group on single-stranded RNAs, releasing 5′ mononucleotides in a sequential manner (175).

This is performed by a unique protein Orn (YjeR), that is inhibited by 3′,5′-adenosine-bisphosphate (pAp), a product of sulfate assimilation and 4-phosphopantetheine formation (176; 177).

This protein is widespread in living organisms (it has a counterpart with the same role in Eukarya), but is absent from many bacterial clades.

It is duplicated in some cases, as its gene may be carried by plasmids.

This is the case in Pseudomonas plasmid pQBR103, where expression is induced by interaction with plants.

Its origin is not known, but it is unlikely to come from the Proteobacteria (178).

This work also revealed that the orn gene, while essential in E. coli, is not essential in Pseudomonas putida KT2440, suggesting that at least one other enzyme can complement the Orn defect.

In Firmicutes (including Mollicutes), an Orn counterpart has been identified as NrnA (YtqI) (179).

Remarkably NrnA is not only a nanoRNase, but it also hydrolyzes pAp, again associating sulfur metabolism and RNA degradation.

NrnA is not essential in B. subtilis, indicating that the function can be performed by other proteins as well.

A 3′-5′ exonuclease of B. subtilis, YhaM, has been proposed to perform this function (180).

However, this enzyme probably prefers deoxyribonucleotides as substrates (156).

It may nevertheless contribute to degradation of short (but perhaps not very short) RNAs in this organism, as it hydrolyzes RNA molecules in vitro (179).

Finally, RNase J1/J2 5′ to 3′ exonuclease activity is not size-limited: it can go all the way to mononucleotides (145) and thus oligoribonuclease or nanoRNase function may not be strictly essential in organisms containing RNase J.

We considerably lack information about the specificity of the various nanoRNases in terms of nucleotide sequence or length.

This may be particularly important in some circumstances.

Indeed, many Bacteria have genes coding for the so-called GGDEF cyclic di-GMP cyclases (181) and this molecule is hydrolyzed first by a phosphodiesterase, releasing pGpG (182), the fate of which has not been clearly established.

The distribution of Orn and NrnA does not span all organisms, and some (such as Actinobacteria) have both (179).

This is interesting as this may reflect the invasion of the genome of the ancestor of Actinobacteria by one of them (possibly Orn, as Proteobacteria are diderm organisms, and more recent than monoderms (183)) creating a situation where the functional redundancy has not yet been eliminated.

This suggests that there may be some advantage in having multiple proteins with nanoRNase activity.

A role in the hydrolysis of specific oligonucleotides could be a reason for stabilizing redundancy.

Remarkably several clades of Bacteria have neither Orn nor NrnA (the alpha-Proteobacteria in particular), which shows that some other protein has been recruited to perform this essential function (FIG. 2).

This is also consistent with the observation that while orn is not essential in P. putida, one does not find obvious counterparts of either nrnA or yhaM either, indicating that some other gene codes for this function.

Finally there are many candidate exonucleases/phosphodiesterases in bacterial genomes, in particular those of the DHH/DHHA1 family (184) (Table II).

It is however difficult, in the absence of experimental data, to predict whether they have DNA or RNA as substrates.

The proteins listed in Table II do not have counterparts in P. putida, showing that, even if some are involved in nanoRNA degradation, there still remain other polypeptides to be discovered that are endowed with this activity.

### Ribonucleases of the Cenome

RNA is ubiquitous.

It is involved in central processes of the cell, which are encoded by the paleome.

It is also an essential component of horizontal gene transfer, in particular via expression of bacteriophage functions.

Besides its role as a template, RNA is also a nutrient supply.

These specific roles, typical of what can be found in the occupation of a particular niche, are encoded by the highly variable part of the genome, the cenome.

Functions related to plasmid and phage expression would be cytoplasmic when expressed under specific conditions (e.g. the onset of the lytic phase for bacteriophages), while functions related to scavenging are most likely to be exported, as they would most probably be highly deleterious in the cell, unless associated with specific inhibitors.

### Exported Ribonucleases

RNase I (Rna, RnsA) of E. coli belongs to the RNase T2/S-RNase group of non-specific endoribonucleases (185).

It is an exported protein, absent from the majority of organisms, but with a counterpart present in individual species here and there, such as in the Cyanobacteria, e.g. Trichodesmium erythreum.

It is present in few species of the beta-Proteobacteria (Chromobacterium violaceum and Thiobacillus denitrificans).

In the gamma-Proteobacteria, it is present in Pasteurellales, in some Enterobacteria: in Shewanella oneidensis and S. baltica, as well as in Methylococcus capsulatus, Legionella pneumophila, Photobacterium profondum.

It is widespread in the alpha-Proteobacteria, where it appears to have evolved fast and this could be an indication that these bacteria are the original niche of the enzyme.

BaRNase (pronounced <<barn-ase>>) is another exported RNase, with guanylspecificity.

It is present in some Firmicutes (Bacillus pumilus, Clostridium acetobutylicum) and considerably evolved in Nocardia farcinica, and in the Alteromonadales, Marinobacter aquaeolei.

Identified in Bacillus amyloliquifaciens, it is not present in its close homolog B. subtilis.

In Enterobacteria, it is present in Yersinia pseudotuberculosis and Y. pestis, Serratia proteomaculans and Photorhabdus luminescens i.e. in members of the family that do not code for RNase I.

It is also present in the delta-Proteobacteria, in Geobacter metallidurens.

The ribonuclease activity of BaRNase is controlled by an inhibitor, barstar, which is present together with the protein in some organisms, but remains alone in others (such as B. subtilis) suggesting that the couple BaRNase/barstar evolved via sequential loss of the genes, with that coding for the ribonuclease lost before that of the inhibitor (3).

This is reminiscent of the way restriction/modification systems evolve, with methylases being conserved when restriction has disappeared (186).

RNase Bsn (YurI) is another exported nuclease, present in a very limited number of species: some Firmicutes (B. subtilis and related species, B. pumilus and Oceanobacillus iheyensis, possibly in Geobacillus kaustophilus) and in a variety of Proteobacteria, where it is probably a DNase with limited similarity (3).

It may also be present in D. radiodurans.

Here again the distribution of the protein is consistent with repeated horizontal gene transfer.

The actual specificity of the protein both in terms of sequence and of the nature of the substrate nucleic acid (RNA or DNA) cannot easily be predicted at this point, in the absence of complementary experimental data.

The YhcR protein is a large exported B. subtilis nonspecific endonuclease (it cleaves both DNA and RNA), with a unique domain structure (the OB-fold), typically found in the thermonuclease family.

It is likely to be secreted by the twin-arginine secretion system, as it possesses a twin-arginine signal peptide, but it appears to have some activity in the cytoplasm as it can complement a multiple RNase defect (187).

As a large protein, presumably with the same function, it is conserved in B. pumilus and O. iheyensis.

It is not possible, however, in the absence of experimental data, to make inferences about the function of the many related proteins that have the same 5′-nucleotidase fold.

### Plasmid and Phage Ribonucleases

RNA is essential for the expression of many horizontally transferred elements, phage and plasmids in particular.

In the latter, suicidal toxin-antitoxin systems are often mediated by RNase activities, associated with a specific inhibitor (188).

The E. coli ChpAI(MazE)-ChpAK(MazF) toxin-antitoxin system (189), for example, consists of an endoribonuclease (and its specific inhibitor) that cleaves target sequences at the 5′ side of A residues in 5′-NAC-3′ sequences (where N is preferentially U or A) (190).

There are five such systems in E. coli K12, presumably acquired by horizontal gene transfer.

This type of system, which can be considered “selfish”, is also well poised to regulate apoptosis in bacteria, often in a ppGpp-dependent manner, to permit some cells in a population to survive in an unfavourable environment.

The role of chromsomally encoded toxin-antitoxin systems in apoptosis is the subject of some controversy.

We have seen that ribosome stalling results in endonucleolytic cleavage, followed by tmRNA mediated degradation of the truncated mRNA and of the truncated proteins it encodes.

Recent work in E. coli has shown that none of its toxin/antitoxin RNase systems is involved in this process (152).

Several related systems have been also identified under different names e.g. Endo A, PemK (191; 192).

The presence of these systems spans a large part of the bacterial tree.

As there is no straightforward counterpart yet identified in reference Bacteria growing at high temperature, it may be absent from these organisms.

They appear also to be lacking in obligate intracellular parasites (193).

Many RNases could be coded by prophages.

In E. coli RNase LS (RnlA, YfjN) is an endonuclease coded in the CP4-57 prophage region of the genome (194).

A mutation in rnlA differentially reduced the decay rate of many E. coli mRNAs, while a 307-nucleotide fragment corresponding to an internal fragment of 23S rRNA accumulated to a high level (194).

This protein appears to be almost unique to E. coli: a counterpart exists in Photobacterium profondum, and perhaps in Desulfotalea psychrophila.

However, careful exploration of other proteomes suggests that the protein could be related to the putative metal-dependent phosphohydrolases present in many genomes.

This could fit well with a phage origin, as these parasites evolve extremely fast.

Several putative phage endonucleases such as YokF or YncB could act on DNA or RNA substrates.

Three classes of RNA molecules organize bacterial cell life.

Stable RNAs (essentially ribosomal RNA and transfer RNA) form the core of the gene expression machinery.

RNAs with an intermediate life time, small regulatory RNAs (srRNAs) and riboswitches are essential regulators whose fundamental importance was only recently uncovered.

Finally messenger RNAs, usually with a short life time, are the intermediates between genes and their final protein products.

All three cases of interact with specific RNases.

In the first class, we find RNases essential for shaping the RNAs into their final active form.

The corresponding functions are therefore ubiquitous.

However, several types of protein folds have been recruited to perform them: this is particularly true when one considers the two model bacteria E. coli and B. subtilis, which do not appear to share many of the enzymes performing the required functions.

These functions form the core of the essential genes of the paleome (paleome 1) involved in RNA degradation (FIG. 1).

Interestingly more functions have been explicitly identified in B. subtilis than in E. coli.

This suggests that a co-evolution pattern of genes in the latter organism should be explored in more detail, in particular to make inferences about the putative function of genes still devoid of a biochemically identified function.

In the second class, we find RNases involved in maintenance, allowing active RNAs to persist in cells in an active form only.

They are expected to be active on misfolded stable RNAs, but also on srRNAs and as controllers of riboswitch states (on/off).

This functional category is extremely important during transitions from one type of environment to another (and this is particularly the case during the universal transitions from exponential growth to stationary phase, and from stationary phase to exponential growth).

The actual experimental analysis of this function is particularly difficult as it is not obviously amenable to biochemical experiments (152).

Here physiology, combined with in silico analysis of genomes, provides some much-wanted information.

While these functions are obviously essential to perpetuate life, there are not strictly indispensable when the cell's environment is stable and provides most of the basic building blocks of the cell.

We therefore expect these genes to belong to the second half of the paleome (paleome 2, FIG. 1).

Also, as conditions are particularly constant for bacteria that have become obligate parasites, it is expected that many of the corresponding functions will be among those which are generally persistent, but often missing in obligate parasites.

Finally, gene expression is regulated in Bacteria by the combination of mRNA levels and mRNA translation control.

The standard pervasive view is that mRNA levels are controlled at the level of transcription initiation; however, this is only meaningful if mRNA degradation is fast.

Furthermore, the type of degradation is important, as the way ribosomes pull mRNA from its DNA template and translate it is essential.

In this context, RNA degradation plays a major role in gene expression.

This can be witnessed by the frequent discrepancy between proteome and transcriptome expression data (195).

33 Different evolution patterns are expected for these three classes of activities.

In particular, regulation is probably a most variable process in the course of evolution.

One expects therefore that mRNA degradation will follow different paths in different species and the most interesting phylogenetic studies will probably deal with specific bacterial clades, associated with specific niches.

By contrast, management of stable RNAs is an essential process, that will remain stable, until a major horizontal gene transfer will bring together several types of RNases that work in a concerted way.

This will be a rare event and will concern only large bacterial families.

In this context, the present study emphasized the particular position of Firmicutes: in these organisms, a completely original RNA degradation system co-evolves, which tends to predict the existence of a degradosome-like complex which would comprise elements that differ considerably from the degradosome of the gamma-Proteobacteria.

A most interesting feature of this system is that, as in the gamma-Proteobacteria, it would be associated with energy saving and managing processes (see enzymes of glycolysis in Table I), suggesting the existence of a novel function of major importance to the RNA maintenance and degradation process.

TABLE I
RNases and RNase-related proteins in B. subtilis
and in E. coli identified by their co-evolutionary pattern

| | | Co-evolving |
|---|---|---|
| | RNases and putative RNases | accessory proteins |
| Co-evolution with | RnhB, Rnc, PnpA, Rnr | YpfD, SmpB, Eno, |
| 16S rRNA | | TpiA, Pgk, GapA, |
| (B. subtilis) | | GlmM, Zwf |
| Co-evolution with | RnmV, MrnC(YazC), YacP, | Cca, CshB(YqfR), |
| RNase J (B. subtilis) | YmdA, YhaM, YkzG, YlbM, | YjbKLMN, YsnB |
| | YlmH, YloA, RnjB(YmfA), | GlcU, GlcT, GlcK, |
| | RnhC(YsgB), NrnA(YtqI), | Pgi, GntK CggR, |
| | YusF, YsnB, YybT | FruR, UgtP, |
| Co-evolution with | Rnr, Rnc, Pnp, RnhA | SpoT, RpsA, Hfq |
| 16S rRNA (E. coli) | | GlpK, Zwf, TpiA, |
| | | Pgk, Eno |
| Co-evolution with | Orn, Rng, Rnd | PcnB, Cca, RelA, |
| RNase E (E. coli) | | Ppx, YhcM |
| | | PykA, Glk |

TABLE II
Proteins of the DHH/DHHA1 family in some organisms.
The AlaS and RecJ proteins are highly conserved, while various members of this family are
present in many Bacteria.
Proteins where the function has not been yet identified are not homologous to each other.

| B. subtilis | E. coli | A. baylyi | H. pylori | H. arsenicoxydans | B. Quintana | C. glutamicum |
|---|---|---|---|---|---|---|
| AlaS | AlaS | AlaS | AlaS | AlaS | AlaS | AlaS |
| RecJ | RecJ | RecJ | RecJ | RecJ | RecJ | |
| PpaC | | | | | | |
| NrnA | | | | | | |
| YngD, | | | HP0425, | CGR_1812 | | |
| YorK, | | | HP1042, | | | |
| YybT | | | HP1410 | | | |

### REFERENCES

Aguilaniu H, Gustafsson L, Rigoulet M et al (2003) Asymmetric inheritance of oxidatively damaged proteins during cytokinesis. Science 299:1751-1753.
Ashida H, Danchin A and Yokota A (2005) Was photosynthetic RuBisCO recruited by acquisitive evolution from RuBisCO-like proteins involved in sulfur metabolism? Res Microbiol 156:611-618.
Baba T, Ara T, Hasegawa M et al (2006) Construction of Escherichia coli K-12 in-frame, single-gene knockout mutants: the Keio collection. Mol Syst Biol 2:2006 0008.
Bennett C (1988) Notes on the history of reversible computation. IBM Journal of research and development 44:270-277.
Blum E, Py B, Carpousis A J et al (1997) Polyphosphate kinase is a component of the Escherichia coli RNA degradosome. Mol Microbiol 26:387-398.
Brosh R M, Jr. and Bohr V A (2007) Human premature aging, DNA repair and RecQ helicases. Nucleic Acids Res 35:7527-7544.
Brown M R and Kornberg A (2004) Inorganic polyphosphate in the origin and survival of species. Proc Natl Acad Sci USA 101:16085-16087.
Burhans W C and Weinberger M (2007) DNA replication stress, genome instability and aging. Nucleic Acids Res 35:7545-7556.
Cairns J, Overbaugh J and Miller S (1988) The origin of mutants. Nature 335:142-145.
Carpousis A J (2007) The RNA degradosome of Escherichia coli: an mRNA-degrading machine assembled on RNase E. Annu Rev Microbiol 61:71-87.
Clarke S (2003) Aging as war between chemical and biochemical processes: protein methylation and the recognition of age-damaged proteins for repair. Ageing Res Rev 2:263-285.
Cover T and Thomas J (1991) Elements of information theory. Wiley, New York
Danchin A (1988) Origin of mutants disputed. Nature 336:527.
Danchin A (1989) Homeotopic transformation and the origin of translation. Prog Biophys Mol Biol 54:81-86.
Danchin A (1993 (2007)) Bacteria are not lamarckian. HAL arXiv: q-bio.GN/0702032:hal-00130797.
Danchin A (2003) The Delphic boat. What genomes tell us. Harvard University Press, Cambridge (Mass, USA)
Danchin A (2007) Archives or palimpsests? Bacterial genomes unveil a scenario for the origin of fife. Biological Theory 2:52-61.
Danchin A (2008) A phylogenetic view of bacterial ribonucleases. Progress in Nucleic acids Research and Molecular Biology (accepted for publication).
Danchin A (2009) Bacteria as computers making computers. FEMS Microbiology Reviews (submitted).
Danchin A, Fang G and Noria S (2007) The extant core bacterial proteome is an archive of the origin of life. Proteomics 7:875-889.
David C L, Keener J and Aswad D W (1999) Isoaspartate in ribosomal protein S11 of Escherichia coli. J Bacteriol 181:2872-2877.
Dyson F J (1985) Origins of life. Cambridge University Press, Cambridge, UK
Fang G, Rocha E and Danchin A (2005) How essential are nonessential genes? Mol Biol Evol 22:2147-2156.
Fraley C D, Rashid M H, Lee S S et al (2007) A polyphosphate kinase 1 (ppk1) mutant of Pseudomonas aeruginosa exhibits multiple ultrastructural and functional defects. Proc Natl Acad Sci USA 104:3526-3531.
Fuentealba L C, Eivers E, Geissert D et al (2008) Asymmetric mitosis: Unequal segregation of proteins destined for degradation. Proc Natl Acad Sci USA.
Galletti P, De Bonis M L, Sorrentino A et al (2007) Accumulation of altered aspartyl residues in erythrocyte proteins from patients with Down's syndrome. Febs J 274:5263-5277.
Gueron M (1978) Enhanced selectivity of enzymes by kinetic proofreading. Am Sci 66:202-208.
Holliday R (2006) Aging is no longer an unsolved problem in biology. Ann N Y Acad Sci 1067:1-9.
Hopfield J J (1974) Kinetic proofreading: a new mechanism for reducing errors in biosynthetic processes requiring high specificity. Proc Natl Acad Sci USA 71:4135-4139.
Ishige K and Noguchi T (2001) Polyphosphate:AMP phosphotransferase and polyphosphate:ADP phosphotransferase activities of Pseudomonas aeruginosa. Biochem Biophys Res Commun 281:821-826.
Ishige K, Zhang H and Kornberg A (2002) Polyphosphate kinase (PPK2), a potent, polyphosphate-driven generator of GTP. Proc Natl Acad Sci USA 99:16684-16688.
Jenkins J and Pickersgill R (2001) The architecture of parallel beta-helices and related folds. Prog Biophys Mol Biol 77:111-175.
Kimura M (1979) Model of effectively neutral mutations in which selective constraint is incorporated. Proc Natl Acad Sci USA 76:3440-3444.
Kimura M and Ota T (1974) On some principles governing molecular evolution. Proc Natl Acad Sci USA 71:2848-2852.
Kirkwood T B and Holliday R (1979) The evolution of ageing and longevity. Proc R Soc Lond B Biol Sci 205:531-546.
Kobayashi K, Ehrlich S D, Albertini A et al (2003) Essential Bacillus subtilis genes. Proc Natl Acad Sci USA 100:4678-4683.
Koyré A (1973) The Astronomical Revolution: Copernicus-Kepler-Borelli. Hermann, Paris; Methuen, London; Cornell University Press, Ithaca, N.Y.,
Lam H, Schofield W B and Jacobs-Wagner C (2006) A landmark protein essential for establishing and perpetuating the polarity of a bacterial cell. Cell 124:1011-1023.
Landauer R (1961) Irreversibility and heat generation in the computing process. IBM Journal of research and development 3:184-191.
Lin-Chao S, Chiou N T and Schuster G (2007) The PNPase, exosome and RNA helicases as the building components of evolutionarily-conserved RNA degradation machines. J Biomed Sci 14:523-532.
Lindner A B, Madden R, Demarez A et al (2008) Asymmetric segregation of protein aggregates is associated with cellular aging and rejuvenation. Proc Natl Acad Sci USA 105:3076-3081.
Livshits G (2005) Genetic epidemiology of skeletal system aging in apparently healthy human population. Mech Ageing Dev 126:269-279.
Mechold U, Fang G, Ngo S et al (2007) YtqI from Bacillus subtilis has both oligoribonuclease and pAp-phosphatase activity. Nucleic Acids Res 35:4552-4561.
Medigue C, Krin E, Pascal G et al (2005) Coping with cold: the genome of the versatile marine Antarctica bacterium Pseudoalteromonas haloplanktis TAC125. Genome Res 15:1325-1335.
Movila A, Uspenskaia I, Toderas I et al (2006) Prevalence of Borrelia burgdorferi sensu lato and Coxiella burnetti in ticks collected in different biocenoses in the Republic of Moldova. International Journal of Medical Microbiology 296:172-176.
Myhill J (1952) Some philosophical implications of mathematical logic. I. Three classes of ideas. The Review of Metaphysics 6:165-198.
Ninio J (1975) Kinetic amplification of enzyme discrimination. Biochimie 57:587-595.
Nystrom T (2003) Conditional senescence in bacteria: death of the immortals. Mol Microbiol 48:17-23.
Nystrom T (2007) A Bacterial Kind of Aging. PLoS Genet 3:e224.
Orgel L E (1963) The maintenance of the accuracy of protein synthesis and its relevance to ageing. Proc Natl Acad Sci USA 49:517-521.
Partridge L (2007) Some highlights of research on aging with invertebrates, 2006-2007. Aging Cell 6:595-598.
Portier C (1980) Isolation of a polynucleotide phosphorylase mutant using a kanamycin resistant determinant. Mol Gen Genet 178:343-349.
Raffaelli N, Finaurini L, Mazzola F et al (2004) Characterization of Mycobacterium tuberculosis NAD kinase: functional analysis of the full-length enzyme by site-directed mutagenesis. Biochemistry 43:7610-7617.
Rattan S I (1996) Synthesis, modifications, and turnover of proteins during aging. Exp Gerontol 31:33-47.
Rattan S I (2008) Increased molecular damage and heterogeneity as the basis of aging. Biol Chem 389:267-272.
Riley M, Staley J T, Danchin A et al (2008) Genomics of an extreme psychrophile, Psychromonas ingrahamii. BMC Genomics 9:210.
Rocha E, Fralick J, Vediyappan G et al (2003) A strand-specific model for chromosome segregation in bacteria. Mol Microbiol 49:895-903.
Schrödinger E (1945) What is life? The physical aspect of the living cell. The Macmillan Company, New York
Shannon C and Weaver W (1949) The Mathematical Theory of Communication. The University of Illinois Press, Urbana (USA)
Shimizu T, Matsuoka Y and Shirasawa T (2005) Biological significance of isoaspartate and its repair system. Biol Pharm Bull 28:1590-1596.
Spencer H (1864) Principles of Biology. Williams and Norgate, London
Steane A (1998) Quantum Computing. Reports on Progress in Physics 61:117-173.
Stearns S (1992) The Evolution of Life Histories. Oxford University Press, Oxford
Sterelny K (2001) Dawkins vs. Gould: Survival of the fittest. Icon Books, Cambridge (UK)
Stewart E and Taddei F (2005) Aging in Escherichia coli: signals in the noise. Bioessays 27:983.
Stewart E J, Madden R, Paul G et al (2005) Aging and death in an organism that reproduces by morphologically symmetric division. PLoS Biol 3:e45.
Szathmary E (2000) The evolution of replicators. Philos Trans R Soc Lond B Biol Sci 355:1669-1676.
Szathmary E (2006) The origin of replicators and reproducers. Philos Trans R Soc Lond B Biol Sci 361:1761-1776.
Szilard L (1929) Uber die Entropieverminderung in einem thermodynamischen System be eingriffen intelligenter Wesen. Zeitchrift fur Physik 53:840-856.
Thompson L W and Krawiec S (1983) Acquisitive evolution of ribitol dehydrogenase in Klebsiella pneumoniae. J Bacteriol 154:1027-1031.
Yamamoto A, Takagi H, Kitamura D et al (1998) Deficiency in protein L-isoaspartyl methyltransferase results in a fatal progressive epilepsy. J Neurosci 18:2063-2074.
Yockey H P (1992) Information theory and molecular biology. Cambridge University Press, Cambridge (UK)
Zarivach R, Deng W, Vuckovic M et al (2008) Structural analysis of the essential self-cleaving type III secretion proteins EscU and SpaS. Nature 453:124-127.
Zhang H, Ishige K and Kornberg A (2002) A polyphosphate kinase (PPK2) widely conserved in bacteria. Proc Natl Acad Sci USA 99:16678-16683.
1. Alpers, D. H., and Tomkins, G. M. (1965). The order of induction and deinduction of the enzymes of the lactose operon in E. coli. Proc Natl Acad Sci USA 53, 797-802.
2. Dobzhansky, T. (1964). Biology, Molecular and Organismic. Am Zool 4, 443-452.
3. Condon, C., and Putzer, H. (2002). The phylogenetic distribution of bacterial ribonucleases. Nucleic Acids Res 30, 5339-5346.
4. Thompson, L. W., and Krawiec, S. (1983). Acquisitive evolution of ribitol dehydrogenase in Klebsiella pneumoniae. J Bacteriol 154, 1027-1031.
5. Ashida, H., Danchin, A., and Yokota, A. (2005). Was photosynthetic RuBisCO recruited by acquisitive evolution from RuBisCO-like proteins involved in sulfur metabolism? Res Microbiol 156, 611-618.
6. Allen, C., Bekoff, M., and Lauder, G., Eds. (1998). “Nature's Purposes”. MIT Press, Cambridge, Mass.
7. Bernal, J. D. (1951). “The physical basis of life”. Routledge and Kegan Paul, London.
8. Granick, S. (1957). Speculations on the origin and evolution of photosynthesis. Annals New York Acad. Sci. 69, 292-308.
9. Cairns-Smith, A. (1982). “Genetic takeover and the mineral origin of life”. Cambridge University Press, Cambridge (UK).
10. Wachtershauser, G. (1988). Before enzymes and templates: theory of surface metabolism. Microbiol Rev 52, 452-484.
11. Danchin, A. (1989). Homeotopic transformation and the origin of translation. Prog Biophys Mol Biol 54, 81-86.
2. Fang, G., Rocha, E., and Danchin, A. (2005). How essential are nonessential genes? Mol Biol Evol 22, 2147-2156.
13. Danchin, A., Fang, G., and Noria, S. (2007). The extant core bacterial proteome is an archive of the origin of life. Proteomics 7, 875-889.
14. Lawrence, J. G., and Roth, J. R. (1996). Selfish operons: horizontal transfer may drive the evolution of gene clusters. Genetics 143, 1843-1860.
15. Fang, G., Rocha, E. P., and Danchin, A. (2008). Persistence drives gene clustering in bacterial genomes. BMC Genomics 9, 4.
16. Chen, X., Li, N., and Ellington, A. D. (2007). Ribozyme catalysis of metabolism in the RNA world. Chem Biodivers 4, 633-655.
17. Altman, S., Wesolowski, D., Guerrier-Takada, C., and Li, Y. (2005). RNase P cleaves transient structures in some riboswitches. Proc Natl Acad Sci USA 102, 11284-11289.
18. Ko, J. H., and Altman, S. (2007). OLE RNA, an RNA motif that is highly conserved in several extremophilic bacteria, is a substrate for and can be regulated by RNase P RNA. Proc Natl Acad Sci USA 104, 7815-7820.
19. Altman, S. (1989). Ribonuclease P: an enzyme with a catalytic RNA subunit. Adv Enzymol Relat Areas Mol Biol 62, 1-36.
20. Willkomm, D. K., Feltens, R., and Hartmann, R. K. (2002). tRNA maturation in Aquifex aeolicus. Biochimie 84, 713-722.
21. Willkomm, D. K., Minnerup, J., Huttenhofer, A., and Hartmann, R. K. (2005). Experimental RNomics in Aquifex aeolicus: identification of small non-coding RNAs and the putative 6S RNA homolog. Nucleic Acids Res 33, 1949-1960.
22. Randau, L., Schroder, I., and Soll, D. (2008). Life without RNase P. Nature 453, 120-123.
23. Marszalkowski, M., Willkomm, D. K., and Hartmann, R. K. (2008). 5′-End maturation of tRNA in Aquifex aeolicus. Biol Chem (in press).
24. Frank, D. N., and Pace, N. R. (1998). Ribonuclease P: unity and diversity in a tRNA processing ribozyme. Annu Rev Biochem 67, 153-180.
25. Guerrier-Takada, C., Lumelsky, N., and Altman, S. (1989). Specific interactions in RNA enzyme-substrate complexes. Science 246, 1578-1584.
6. Lundberg, U., and Altman, S. (1995). Processing of the precursor to the catalytic RNA subunit of RNase P from Escherichia coli. Rna 1, 327-334.
27. Gossringer, M., Kretschmer-Kazemi Far, R., and Hartmann, R. K. (2006). Analysis of RNase P protein (RnpA) expression in Bacillus subtilis utilizing strains with suppressible rnpA expression. J Bacteriol 188, 6816-6823.
28. Waugh, D. S., and Pace, N. R. (1990). Complementation of an RNase P RNA (rnpB) gene deletion in Escherichia coli by homologous genes from distantly related eubacteria. J Bacteriol 172, 6316-6322.
29. Massire, C., Jaeger, L., and Westhof, E. (1998). Derivation of the three-dimensional architecture of bacterial ribonuclease P RNAs from comparative sequence analysis. J Mol Biol 279, 773-793.
30. Wegscheid, B., Condon, C., and Hartmann, R. K. (2006). Type A and B RNase P RNAs are interchangeable in vivo despite substantial biophysical differences. EMBO Rep 7, 411-417.
31. Loria, A., and Pan, T. (1996). Domain structure of the ribozyme from eubacterial ribonuclease P. Rna 2, 551-563.
32. Kazantsev, A. V., and Pace, N. R. (2006). Bacterial RNase P: a new view of an ancient enzyme. Nat Rev Microbiol 4, 729-740.
33. Loria, A., and Pan, T. (2001). Modular construction for function of a ribonucleoprotein enzyme: the catalytic domain of Bacillus subtilis RNase P complexed with B. subtilis RNase P protein. Nucleic Acids Res 29, 1892-1897.
34. Haas, E. S., and Brown, J. W. (1998). Evolutionary variation in bacterial RNase P RNAs. Nucleic Acids Res 26, 4093-4099.
35. Urbonavicius, J., Brochier-Armanet, C., Skouloubris, S., Myllykallio, H., and Grosjean, H. (2007). In vitro detection of the enzymatic activity of folatedependent tRNA (Uracil-54,-C5)-methyltransferase: evolutionary implications. Methods Enzymol 425, 103-119.
36. Pitulle, C., Strehse, C., Brown, J. W., and Breitschwerdt, E. B. (2002). Investigation of the phylogenetic relationships within the genus Bartonella based on comparative sequence analysis of the rnpB gene, 16S rDNA and 23S rDNA. Int J Syst Evol Microbiol 52, 2075-2080.
37. Honda, D., Yokota, A., and Sugiyama, J. (1999). Detection of seven major evolutionary lineages in cyanobacteria based on the 16S rRNA gene sequence analysis with new sequences of five marine Synechococcus strains. J Mol Evol 48, 723-739.
38. Herrmann, B., Pettersson, B., Everett, K. D., Mikkelsen, N. E., and Kirsebom, L. A. (2000). Characterization of the rnpB gene and RNase P RNA in the order Chlamydiales. Int J Syst Evol Microbiol 50 Pt 1, 149-158.
39. Griffiths, E., and Gupta, R. S. (2004). Signature sequences in diverse proteins provide evidence for the late divergence of the Order Aquificales. Int Microbiol 7, 41-52.
40. Marszalkowski, M., Teune, J. H., Steger, G., Hartmann, R. K., and Willkomm, D. K. (2006). Thermostable RNase P RNAs lacking P18 identified in the Aquificales. RNA 12, 1915-1921.
41. Fuerst, J. A. (1995). The planctomycetes: emerging models for microbial ecology, evolution and cell biology. Microbiology 141 (Pt 7), 1493-1506.
42. Butler, M. K., and Fuerst, J. A. (2004). Comparative analysis of ribonuclease P RNA of the planctomycetes. Int J Syst Evol Microbiol 54, 1333-1344.
43. Cilia, V., Lafay, B., and Christen, R. (1996). Sequence heterogeneities among 16S ribosomal RNA sequences, and their effect on phylogenetic analyses at the species level. Mol Biol Evol 13, 451-461.
44. Acinas, S. G., Marcelino, L. A., Klepac-Ceraj, V., and Polz, M. F. (2004). Divergence and redundancy of 16S rRNA sequences in genomes with multiple rrn operons. J Bacteriol 186, 2629-2635.
45. Zawilak, A., Cebrat, S., Mackiewicz, P., Krol-Hulewicz, A., Jakimowicz, D., Messer, W., Gosciniak, G., and Zakrzewska-Czerwinska, J. (2001). Identification of a putative chromosomal replication origin from Helicobacter pylori and its interaction with the initiator protein DnaA. Nucleic Acids Res 29, 2251-2259.
46. Hall, T. A., and Brown, J. W. (2001). The ribonuclease P family. Methods Enzymol 341, 56-77.
47. Kazantsev, A. V., Krivenko, A. A., Harrington, D. J., Carter, R. J., Holbrook, S. R., Adams, P. D., and Pace, N. R. (2003). High-resolution structure of RNase P protein from Thermotoga maritima. Proc Natl Acad Sci USA 100, 7497-7502.
48. Niranjanakumari, S., Day-Storms, J. J., Ahmed, M., Hsieh, J., Zahler, N. H., Venters, R. A., and Fierke, C. A. (2007). Probing the architecture of the B. subtilis RNase P holoenzyme active site by cross-linking and affinity cleavage. Rna 13, 521-535.
49. Baba, T., Ara, T., Hasegawa, M., Takai, Y., Okumura, Y., Baba, M., Datsenko, K. A., Tomita, M., Wanner, B. L., and Mori, H. (2006). Construction of Escherichia coli K-12 in-frame, single-gene knockout mutants: the Keio collection. Mol Syst Biol 2, 2006 0008.
50. Kobayashi, K., Ehrlich, S. D., Albertini, A., Amati, G., Andersen, K. K., Arnaud, M., Asai, K., Ashikaga, S., Aymerich, S., Bessieres, P., Boland, F., Brignell, S. C., Bron, S., Bunai, K., Chapuis, J., Christiansen, L. C., Danchin, A., Debarbouille, M., Dervyn, E., Deuerling, E., Devine, K., Devine, S. K., Dreesen, O., Errington, J., Fillinger, S., Foster, S. J., Fujita, Y., Galizzi, A., Gardan, R., Eschevins, C., Fukushima, T., Haga, K., Harwood, C. R., Hecker, M., Hosoya, D., Hullo, M. F., Kakeshita, H., Karamata, D., Kasahara, Y., Kawamura, F., Koga, K., Koski, P., Kuwana, R., Imamura, D., Ishimaru, M., Ishikawa, S., Ishio, I., Le Coq, D., Masson, A., Mauel, C., Meima, R., Mellado, R. P., Moir, A., Moriya, S., Nagakawa, E., Nanamiya, H., Nakai, S., Nygaard, P., Ogura, M., Ohanan, T., O'Reilly, M., O'Rourke, M., Pragai, Z., Pooley, H. M., Rapoport, G., Rawlins, J. P., Rivas, L. A., Rivolta, C., Sadaie, A., Sadaie, Y., Sarvas, M., Sato, T., Saxild, H. H., Scanlan, E., Schumann, W., Seegers, J. F., Sekiguchi, J., Sekowska, A., Seror, S. J., Simon, M., Stragier, P., Studer, R., Takamatsu, H., Tanaka, T., Takeuchi, M., Thomaides, H. B., Vagner, V., van Dijl, J. M., Watabe, K., Wipat, A., Yamamoto, H., Yamamoto, M., Yamamoto, Y., Yamane, K., Yata, K., Yoshida, K., Yoshikawa, H., Zuber, U., and Ogasawara, N. (2003). Essential Bacillus subtilis genes. Proc Natl Acad Sci USA 100, 4678-4683.
51. Kimura, M., and Ota, T. (1974). On some principles governing molecular evolution. Proc Natl Acad Sci USA 71, 2848-2852.
52. Qian, H. (2006). Reducing intrinsic biochemical noise in cells and its thermodynamic limit. J Mol Biol 362, 387-392.
53. Carpousis, A. J. (2007). The RNA degradosome of Escherichia coli: an mRNA degrading machine assembled on RNase E. Annu Rev Microbiol 61, 71-87.
54. Evguenieva-Hackenberg, E., Walter, P., Hochleitner, E., Lottspeich, F., and Klug, G. (2003). An exosome-like complex in Sulfolobus solfataricus. EMBO Rep 4, 889-893.
55. Rauhut, R., and Klug, G. (1999). mRNA degradation in bacteria. FEMS Microbiol Rev 23, 353-370.
56. Frazao, C., McVey, C. E., Amblar, M., Barbas, A., Vonrhein, C., Arraiano, C. M., and Carrondo, M. A. (2006). Unravelling the dynamics of RNA degradation by ribonuclease II and its RNA-bound complex. Nature 443, 110-114.
57. Henaut, A., Lisacek, F., Nitschke, P., Moszer, I., and Danchin, A. (1998). Global analysis of genomic texts: the distribution of AGCT tetranucleotides in the Escherichia coli and Bacillus subtilis genomes predicts translational frameshifting and ribosomal hopping in several genes. Electrophoresis 19, 515-527.
58. Johnstone, S. A., Waisman, D. M., and Rattner, J. B. (1992). Enolase is present at the centrosome of HeLa cells. Exp Cell Res 202, 458-463.
59. Xi, J. H., Bai, F., McGaha, R., and Andley, U. P. (2006). Alpha-crystallin expression affects microtubule assembly and prevents their aggregation. Faseb J 20, 846-857.
60. Keller, A., Peltzer, J., Carpentier, G., Horvath, I., Olah, J., Duchesnay, A., Orosz, F., and Ovadi, J. (2007). Interactions of enolase isoforms with tubulin and microtubules during myogenesis. Biochim Biophys Acta 1770, 919-926.
61. Kovacs, L., Csanadi, A., Megyeri, K., Kaberdin, V. R., and Miczak, A. (2005). Mycobacterial RNase E-associated proteins. Microbiol. Immunol 49, 1003-1007.
62. Blum, E., Py, B., Carpousis, A. J., and Higgins, C. F. (1997). Polyphosphate kinase is a component of the Escherichia coli RNA degradosome. Mol Microbiol 26, 387-398.
63. Jasiecki, J., and Wegrzyn, G. (2005). Localization of Escherichia coli poly(A) polymerase I in cellular membrane. Biochem Biophys Res Commun 329, 598-602.
64. Danchin, A. (1997). Comparison between the Escherichia coli and Bacillus subtilis genomes suggests that a major function of polynucleotide phosphorylase is to synthesize CDP. DNA Res 4, 9-18.
65. Feng, Y., Huang, H., Liao, J., and Cohen, S, N. (2001). Escherichia coli poly(A)-binding proteins that interact with components of degradosomes or impede RNA decay mediated by polynucleotide phosphorylase and RNase E. J Biol Chem 276, 31651-31656.
66. Nitschke, P., Guerdoux-Jamet, P., Chiapello, H., Faroux, G., Henaut, C., Henaut, A., and Danchin, A. (1998). Indigo: a World-Wide-Web review of genomes and gene functions. FEMS Microbiol Rev 22, 207-227.
67. Bailly-Bechet, M., Danchin, A., Iqbal, M., Marsili, M., and Vergassola, M. (2006). Codon usage domains over bacterial chromosomes. PLoS Comput Biol 2, e37.
68. Lipmann, F. (1941 (2006)). Metabolic Generation and Utilization of Phosphate Bond Energy. In “Advances in Enzymology and Related Areas of Molecular Biology” (C. H. W. F. F. Nord, ed.), Vol. 1, pp. 99-162. Fordham University, New York, N.Y.
69. Danchin, A. (2008). Natural Selection and Immortality. Biogerontology (submitted).
70. Ollagnier, S., Mulliez, E., Gaillard, J., Eliasson, R., Fontecave, M., and Reichard, P. (1996). The anaerobic Escherichia coli ribonucleotide reductase. Subunit structure and iron sulfur center. J Biol Chem 271, 9410-9416.
71. Cohen, S. S. (1960). A hypothesis on a possible competitive relation between DNA synthesis and protein synthesis. Cancer Res 20, 698-699.
72. Rocha, E. P., and Danchin, A. (2002). Base composition bias might result from competition for metabolic resources. Trends Genet. 18, 291-294.
73. Symmons, M. F., Jones, G. H., and Luisi, B. F. (2000). A duplicated fold is the structural basis for polynucleotide phosphorylase catalytic activity, processivity, and regulation. Structure 8, 1215-1226.
74. Yehudai-Resheff, S., Portnoy, V., Yogev, S., Adir, N., and Schuster, G. (2003). Domain analysis of the chloroplast polynucleotide phosphorylase reveals discrete functions in RNA degradation, polyadenylation, and sequence homology with exosome proteins. Plant Cell 15, 2003-2019.
75. Lee, K., and Cohen, S, N. (2003). A Streptomyces coelicolor functional orthologue of Escherichia coli RNase E shows shuffling of catalytic and PNPasebinding domains. Mol Microbiol 48, 349-360.
76. Bermudez-Cruz, R. M., Fernandez-Ramirez, F., Kameyama-Kawabe, L., and Montanez, C. (2005). Conserved domains in polynucleotide phosphorylase among eubacteria. Biochimie 87, 737-745.
77. Boutet, E., Lieberherr, D., Tognolli, M., Schneider, M., and Bairoch, A. (2007). UniProtKB/Swiss-Prot: The Manually Annotated Section of the UniProt KnowledgeBase. Methods Mol Biol 406, 89-112.
78. Kelly, K. O., and Deutscher, M. P. (1992). Characterization of Escherichia coli RNase PH. J Biol Chem 267, 17153-17158.
79. Li, Z., and Deutscher, M. P. (1996). Maturation pathways for E. coli tRNA precursors: a random multienzyme process in vivo. Cell 86, 503-512.
80. Ishii, R., Nureki, O., and Yokoyama, S. (2003). Crystal structure of the tRNA processing enzyme RNase PH from Aquifex aeolicus. J Biol Chem 278, 32397-32404.
81. Harlow, L. S., Kadziola, A., Jensen, K. F., and Larsen, S. (2004). Crystal structure of the phosphorolytic exoribonuclease RNase PH from Bacillus subtilis and implications for its quaternary structure and tRNA binding. Protein Sci 13, 668-677.
82. Wen, T., Oussenko, I. A., Pellegrini, O., Bechhofer, D. H., and Condon, C. (2005). Ribonuclease PH plays a major role in the exonucleolytic maturation of CCA-containing tRNA precursors in Bacillus subtilis. Nucleic Acids Res 33, 3636-3643.
83. Li, Z., Pandit, S., and Deutscher, M. P. (1998). 3′ exoribonucleolytic trimming is a common feature of the maturation of small, stable RNAs in Escherichia coli. Proc Natl Acad Sci USA 95, 2856-2861.
84. Lizano, E., Scheibe, M., Rammelt, C., Betat, H., and Morl, M. (2008). A comparative analysis of CCA-adding enzymes from human and E. coli: Differences in CCA addition and tRNA 3′-end repair. Biochimie.
85. Lin-Chao, S., Chiou, N. T., and Schuster, G. (2007). The PNPase, exosome and RNA helicases as the building components of evolutionarily-conserved RNA degradation machines. J Biomed Sci 14, 523-532.
86. Jones, G. H., and Bibb, M. J. (1996). Guanosine pentaphosphate synthetase from Streptomyces antibioticus is also a polynucleotide phosphorylase. J Bacteriol 178, 4281-4288.
87. Kuroda, A., Murphy, H., Cashel, M., and Kornberg, A. (1997). Guanosine tetraand pentaphosphate promote accumulation of inorganic polyphosphate in Escherichia coli. J Biol Chem 272, 21240-21243.
88. Sengupta, J., Agrawal, R. K., and Frank, J. (2001). Visualization of protein S1 within the 30S ribosomal subunit and its interaction with messenger RNA. Proc Natl Acad Sci USA 98, 11991-11996.
89. Frangeul, L., Nelson, K. E., Buchrieser, C., Danchin, A., Glaser, P., and Kunst, F. (1999). Cloning and assembly strategies in microbial genome projects. Microbiology 145 (Pt 10), 2625-2634.
90. Bordeau, V., and Felden, B. (2002). Ribosomal protein S1 induces a conformational change of tmRNA; more than one protein S1 per molecule of tmRNA. Biochimie 84, 723-729.
91. Saguy, M., Gillet, R., Skorski, P., Hermann-Le Denmat, S., and Felden, B. (2007). Ribosomal protein S1 influences trans-translation in vitro and in vivo. Nucleic Acids Res 35, 2368-2376.
92. Amblar, M., Barbas, A., Fialho, A. M., and Arraiano, C. M. (2006). Characterization of the functional domains of Escherichia coli RNase II. J Mol Biol 360, 921-933.
93. Schubert, M., Edge, R. E., Lario, P., Cook, M. A., Strynadka, N. C., Mackie, G. A., and McIntosh, L. P. (2004). Structural characterization of the RNase E S1 domain and identification of its oligonucleotide-binding and dimerization interfaces. J Mol Biol 341, 37-54.
94. Durand, S., Richard, G., Bisaglia, M., Laalami, S., Bontems, F., and Uzan, M. (2006). Activation of RegB endoribonuclease by S1 ribosomal protein requires an 11 nt conserved sequence. Nucleic Acids Res 34, 6549-6560.
95. Odaert, B., Saida, F., Aliprandi, P., Durand, S., Crechet, J. B., Guerois, R., Laalami, S., Uzan, M., and Bontems, F. (2007). Structural and functional studies of RegB, a new member of a family of sequence-specific ribonucleases involved in mRNA inactivation on the ribosome. J Biol Chem 282, 2019-2028.
96. Karlin, S., Brocchieri, L., Mrazek, J., and Kaiser, D. (2006). Distinguishing features of delta-proteobacterial genomes. Proc Natl Acad Sci USA 103, 11352-11357.
97. Grishin, N. V. (2001). KH domain: one motif, two folds. Nucleic Acids Res 29, 638-643.
98. Inoue, K., Chen, J., Tan, Q., and Inouye, M. (2006). Era and RbfA have overlapping function in ribosome biogenesis in Escherichia coli. J Mol Microbiol Biotechnol 11, 41-52.
99. Heeb, S., Kuehne, S. A., Bycroft, M., Crivii, S., Allen, M. D., Haas, D., Camara, M., and Williams, P. (2006). Functional analysis of the post-transcriptional regulator RsmA reveals a novel RNA-binding site. J Mol Biol 355, 1026-1036.
100. Stickney, L. M., Hankins, J. S., Miao, X., and Mackie, G. A. (2005). Function of the conserved S1 and KH domains in polynucleotide phosphorylase. J Bacteriol 187, 7214-7221.
101. Andrade, J. M., and Arraiano, C. M. (2008). PNPase is a key player in the regulation of small RNAs that control the expression of outer membrane proteins. RNA 14, 543-551.
102. Lin, P. H., and Lin-Chao, S. (2005). RhlB helicase rather than enolase is the beta-subunit of the Escherichia coli polynucleotide phosphorylase (PNPase)-exoribonucleolytic complex. Proc Natl Acad Sci USA 102, 16590-16595.
103. Khemici, V., Toesca, I., Poljak, L., Vanzo, N. F., and Carpousis, A. J. (2004). The RNase E of Escherichia coli has at least two binding sites for DEAD-box RNA helicases: functional replacement of RhlB by RhlE. Mol Microbiol 54, 1422-1430.
104. Hilleren, P., and Parker, R. (1999). Mechanisms of mRNA surveillance in eukaryotes. Annu Rev Genet. 33, 229-260.
105. Liou, G. G., Chang, H. Y., Lin, C. S., and Lin-Chao, S. (2002). DEAD box RhlB RNA helicase physically associates with exoribonuclease PNPase to degrade double-stranded RNA independent of the degradosome-assembling region of RNase E. J Biol Chem 277, 41157-41162.
106. Koo, J. T., Choe, J., and Moseley, S. L. (2004). HrpA, a DEAH-box RNA helicase, is involved in mRNA processing of a fimbrial operon in Escherichia coli. Mol Microbiol 52, 1813-1826.
107. Burgess, S. M., and Guthrie, C. (1993). Beat the clock: paradigms for NTPases in the maintenance of biological fidelity. Trends Biochem Sci 18, 381-384.
108. Worrall, J. A., Howe, F. S., McKay, A. R., Robinson, C. V., and Luisi, B. F. (2007). Allosteric activation of the ATPase activity of the Escherichia coli RhlB RNA helicase. J Biol. Chem.
109. Russell, R. (2008). RNA misfolding and the action of chaperones. Front Biosci 13, 1-20.
110. Medigue, C., Krin, E., Pascal, G., Barbe, V., Bernsel, A., Bertin, P. N., Cheung, F., Cruveiller, S., D'Amico, S., Duilio, A., Fang, G., Feller, G., Ho, C., Mangenot, S., Marino, G., Nilsson, J., Parrilli, E., Rocha, E. P., Rouy, Z., Sekowska, A., Tutino, M. L., Vallenet, D., von Heijne, G., and Danchin, A. (2005). Coping with cold: the genome of the versatile marine Antarctica bacterium Pseudoalteromonas haloplanktis TAC125. Genome Res 15, 1325-1335.
111. Brown, M. R., and Kornberg, A. (2004). Inorganic polyphosphate in the origin and survival of species. Proc Natl Acad Sci USA 101, 16085-16087.
112. Fraley, C. D., Rashid, M. H., Lee, S. S., Gottschalk, R., Harrison, J., Wood, P. J., Brown, M. R., and Kornberg, A. (2007). A polyphosphate kinase 1 (ppk1) mutant of Pseudomonas aeruginosa exhibits multiple ultrastructural and functional defects. Proc Natl Acad Sci USA 104, 3526-3531.
113. McMahon, K. D., Yilmaz, S., He, S., Gall, D. L., Jenkins, D., and Keasling, J. D. (2007). Polyphosphate kinase genes from full-scale activated sludge plants. Appl Microbiol Biotechnol 77, 167-173.
114. Zhang, H., Ishige, K., and Kornberg, A. (2002). A polyphosphate kinase (PPK2) widely conserved in bacteria. Proc Natl Acad Sci USA 99, 16678-16683.
115. Raffaelli, N., Finaurini, L., Mazzola, F., Pucci, L., Sorci, L., Amici, A., and Magni, G. (2004). Characterization of Mycobacterium tuberculosis NAD kinase: functional analysis of the full-length enzyme by site-directed mutagenesis. Biochemistry 43, 7610-7617.
116. Taghbalout, A., and Rothfield, L. (2007). RNaseE and the other constituents of the RNA degradosome are components of the bacterial cytoskeleton. Proc Natl Acad Sci USA 104, 1667-1672.
117. Noria, S., and Danchin, A. (2002). Uehara Memorial Foundation Symposium: Genome Science: towards a new paradigm?, Tokyo.
118. Chakrabarty, A. M. (1998). Nucleoside diphosphate kinase: role in bacterial growth, virulence, cell signalling and polysaccharide synthesis. Mol Microbiol 28, 875-882.
119. Oslancova, A., and Janecek, S. (2004). Evolutionary relatedness between glycolytic enzymes most frequently occurring in genomes. Folia Microbiol (Praha) 49, 247-258.
120. Scheibe, M., Bonin, S., Hajnsdorf, E., Betat, H., and Morl, M. (2007). Hfq stimulates the activity of the CCA-adding enzyme. BMC Mol Biol 8, 92.
121. Kushner, S. R. (2004). mRNA decay in prokaryotes and eukaryotes: different approaches to a similar problem. IUBMB Life 56, 585-594.
122. Campos-Guillen, J., Bralley, P., Jones, G. H., Bechhofer, D. H., and Olmedo-Alvarez, G. (2005). Addition of poly(A) and heteropolymeric 3′ ends in Bacillus subtilis wild-type and polynucleotide phosphorylase-deficient strains. J Bacteriol 187, 4698-4706.
123. Jourdan, S. S., and McDowall, K. J. (2008). Sensing of 5′ monophosphate by Escherichia coli RNase G can significantly enhance association with RNA and stimulate the decay of functional mRNA transcripts in vivo. Mol Microbiol 67, 102-115.
124. Kaga, N., Umitsuki, G., Nagai, K., and Wachi, M. (2002). RNase G-dependent degradation of the eno mRNA encoding a glycolysis enzyme enolase in Escherichia coli. Biosci Biotechnol Biochem 66, 2216-2220.
125. Okada, Y., Wachi, M., Hirata, A., Suzuki, K., Nagai, K., and Matsuhashi, M. (1994). Cytoplasmic axial filaments in Escherichia coli cells: possible function in the mechanism of chromosome segregation and cell division. J Bacteriol 176, 917-922.
126. Wachi, M., Umitsuki, G., Shimizu, M., Takada, A., and Nagai, K. (1999). Escherichia coli cafA gene encodes a novel RNase, designated as RNase G, involved in processing of the 5′ end of 16S rRNA. Biochem Biophys Res Commun 259, 483-488.
127. Kaberdin, V. R., and Bizebard, T. (2005). Characterization of Aquifex aeolicus RNase E/G. Biochem Biophys Res Commun 327, 382-392.
128. Deana, A., and Belasco, J. G. (2004). The function of RNase G in Escherichia coli is constrained by its amino and carboxyl termihi. Mol Microbiol 51, 1205-1217.
129. Zeller, M. E., Csanadi, A., Miczak, A., Rose, T., Bizebard, T., and Kaberdin, V. R. (2007). Quaternary structure and biochemical properties of mycobacterial RNase E/G. Biochem J 403, 207-215.
130. Britton, R. A., Wen, T., Schaefer, L., Pellegrini, O., Uicker, W. C., Mathy, N., Tobin, C., Daou, R., Szyk, J., and Condon, C. (2007). Maturation of the 5′ end of Bacillus subtilis 16S rRNA by the essential ribonuclease YkqC/RNase J1. Mol Microbiol 63, 127-138.
131. Suzuki, K., Babitzke, P., Kushner, S. R., and Romeo, T. (2006). Identification of a novel regulatory protein (CsrD) that targets the global regulatory RNAs CsrB and CsrC for degradation by RNase E. Genes Dev 20, 2605-2617.
132. Li, H. (2007). Complexes of tRNA and maturation enzymes: shaping up for translation. Curr Opin Struct Biol 17, 293-301.
133. Callaghan, A. J., Marcaida, M. J., Stead, J. A., McDowall, K. J., Scott, W. G., and Luisi, B. F. (2005). Structure of Escherichia coli RNase E catalytic domain and implications for RNA turnover. Nature 437, 1187-1191.
134. Kaberdin, V. R., Miczak, A., Jakobsen, J. S., Lin-Chao, S., McDowall, K. J., and von Gabain, A. (1998). The endoribonucleolytic N-terminal half of Escherichia coli RNase E is evolutionarily conserved in Synechocystis sp. and other bacteria but not the C-terminal half, which is sufficient for degradosome assembly. Proc Natl Acad Sci USA 95, 11637-11642.
135. Lee, K., Zhan, X., Gao, J., Qiu, J., Feng, Y., Meganathan, R., Cohen, S, N., and Georgiou, G. (2003). RraA. a protein inhibitor of RNase E activity that globally modulates RNA abundance in E. coli. Cell 114, 623-634.
136. Gao, J., Lee, K., Zhao, M., Qiu, J., Zhan, X., Saxena, A., Moore, C. J., Cohen, S, N., and Georgiou, G. (2006). Differential modulation of E. coli mRNA abundance by inhibitory proteins that alter the composition of the degradosome. Mol Microbiol 61, 394-406.
137. Bebrone, C. (2007). Metallo-beta-lactamases (classification, activity, genetic organization, structure, zinc coordination) and their superfamily. Biochem Pharmacol 74, 1686-1701.
138. Dominski, Z. (2007). Nucleases of the metallo-beta-lactamase family and their role in DNA and RNA metabolism. Crit Rev Biochem Mol Biol 42, 67-93.
139. Redko, Y., Li de Lasierra-Gallay, I., and Condon, C. (2007). When all's zed and done: the structure and function of RNase Z in prokaryotes. Nat Rev Microbiol 5, 278-286.
140. Pellegrini, O., Nezzar, J., Marchfelder, A., Putzer, H., and Condon, C. (2003). Endonucleolytic processing of CCA-less tRNA precursors by RNase Z in Bacillus subtilis. Embo J 22, 4534-4543.
141. Ezraty, B., Dahlgren, B., and Deutscher, M. P. (2005). The RNase Z homologue encoded by Escherichia coli elaC gene is RNase BN. J Biol Chem 280, 16542-16545.
142. Perwez, T., and Kushner, S. R. (2006). RNase Z in Escherichia coli plays a significant role in mRNA decay. Mol Microbiol 60, 723-737.
143. Vogel, A., Schilling, O., Spath, B., and Marchfelder, A. (2005). The tRNase Z family of proteins: physiological functions, substrate specificity and structural properties. Biol Chem 386, 1253-1264.
144. Even, S., Pellegrini, O., Zig, L., Labas, V., Vinh, J., Brechemmier-Baey, D., and Putzer, H. (2005). Ribonucleases J1 and J2: two novel endoribonucleases in B. subtilis with functional homology to E. coli RNase E. Nucleic Acids Res 33, 2141-2152.
145. Mathy, N., Benard, L., Pellegrini, O., Daou, R., Wen, T., and Condon, C. (2007). 5′-to-3′ exoribonuclease activity in bacteria: role of RNase J1 in rRNA maturation and 5′ stability of mRNA. Cell 129, 681-692.
146. de la Sierra-Gallay, I. L., Zig, L., Jamalli, A., and Putzer, H. (2008). Structural insights into the dual activity of RNase J. Nat Struct Mol Biol 15, 206-212.
147. Evguenieva-Hackenberg, E., and Klug, G. (2000). RNase III processing of intervening sequences found in helix 9 of 23S rRNA in the alpha subclass of Proteobacteria. J Bacteriol 182, 4719-4729.
148. Allsopp, M. T., Van Heerden, H., Steyn, H. C., and Allsopp, B. A. (2003). Phylogenetic relationships among Ehrlichia ruminantium isolates. Ann N Y Acad Sci 990, 685-691.
149. Yamamoto, Y., Sunohara, T., Jojima, K., Inada, T., and Aiba, H. (2003). SsrAmediated trans-translation plays a role in mRNA quality control by facilitating degradation of truncated mRNAs. Rna 9, 408-418.
150. Dulebohn, D., Choy, J., Sundermeier, T., Okan, N., and Karzai, A. W. (2007). Trans-translation: the tmRNA-mediated surveillance mechanism for ribosome rescue, directed protein degradation, and nonstop mRNA &bay. Biochemistry 46, 4681-4693.
151. Richards, J., Mehta, P., and Karzai, A. W. (2006). RNase R degrades non-stop mRNAs selectively in an SmpB-tmRNA-dependent manner. Mol Microbiol 62, 1700-1712.
152. Li, X., Yagi, M., Morita, T., and Aiba, H. (2008). Cleavage of mRNAs and role of tmRNA system under amino acid starvation in Escherichia coli. Mol. Microbiol.
153. Redko, Y., Bechhofer, D. H., and Condon, C. (2008). Mini-III, an unusual member of the RNase III family of enzymes, catalyzes 23S ribosomal RNA maturation in B. subtilis. Molecular Microbiology 68, 1096-1106.
154. Morishita, R., Kawagoshi, A., Sawasaki, T., Madin, K., Ogasawara, T., Oka, T., and Endo, Y. (1999). Ribonuclease activity of rat liver perchloric acid-soluble protein, a potent inhibitor of protein synthesis. J Biol Chem 274, 20688-20692.
155. Hunt, A., Rawlins, J. P., Thomaides, H. B., and Errington, J. (2006). Functional analysis of 11 putative essential genes in Bacillus subtilis. Microbiology 152, 2895-2907.
156. Condon, C. (2003). RNA processing and degradation in Bacillus subtilis. Microbiol Mol Biol Rev 67, 157-174, table of contents.
157. Gattiker, A., Michoud, K., Rivoire, C., Auchincloss, A. H., Coudert, E., Lima, T., Kersey, P., Pagni, M., Sigrist, C. J., Lachaize, C., Veuthey, A. L., Gasteiger, E., and Bairoch, A. (2003). Automated annotation of microbial proteomes in SWISSPROT. Comput Biol Chem 27, 49-58.
158. Callahan, C., and Deutscher, M. P. (1996). Identification and characterization of the Escherichia coli rbn gene encoding the tRNA processing enzyme RNase BN. J Bacteriol 178, 7329-7332.
159. Pascal, G., Medigue, C., and Danchin, A. (2005). Universal biases in protein composition of model prokaryotes. Proteins 60, 27-35.
160. Kochiwa, H., Tomita, M., and Kanai, A. (2007). Evolution of ribonuclease H genes in prokaryotes to avoid inheritance of redundant genes. BMC Evol Biol 7, 128.
161. Ohtani, N., Yanagawa, H., Tomita, M., and Itaya, M. (2004). Identification of the first archaeal Type 1 RNase H gene from Halobacterium sp. NRC-1: archaeal RNase HI can cleave an RNA-DNA junction. Biochem J 381, 795-802.
162. Knizewski, L., and Ginalski, K. (2005). Bacillus subtilis YkuK protein is distantly related to RNase H. FEMS Microbiol Lett 251, 341-346.
163. Vincent, H. A., and Deutscher, M. P. (2006). Substrate recognition and catalysis by the exoribonuclease RNase R. J Biol Chem 281, 29769-29775.
164. Cheng, Z. F., and Deutscher, M. P. (2002). Purification and characterization of the Escherichia coli exoribonuclease RNase R. Comparison with RNase II. J Biol Chem 277, 21624-21629.
165. Purusharth, R. I., Klein, F., Sulthana, S., Jager, S., Jagannadham, M. V., Evguenieva-Hackenberg, E., Ray, M. K., and Klug, G. (2005). Exoribonuclease R interacts with endoribonuclease E and an RNA helicase in the psychrotrophic bacterium Pseudomonas syringae Lz4W. J Biol Chem 280, 14572-14578.
166. Cairrao, F., Chora, A., Zilhao, R., Carpousis, A. J., and Arraiano, C. M. (2001). RNase II levels change according to the growth conditions: characterization of gmr, a new Escherichia coli gene involved in the modulation of RNase II. Mol Microbiol 39, 1550-1561.
167. Cudny, H., and Deutscher, M. P. (1980). Apparent involvement of ribonuclease D in the 3′ processing of tRNA precursors. Proc Natl Acad Sci USA 77, 837-841.
168. Deutscher, M. P., Marlor, C. W., and Zaniewski, R. (1984). Ribonuclease T: new exoribonuclease possibly involved in end-turnover of tRNA. Proc Natl Acad Sci USA 81, 4290-4293.
169. Fukushima, S., Itaya, M., Kato, H., Ogasawara, N., and Yoshikawa, H. (2007). Reassessment of the in vivo functions of DNA polymerase I and RNase H in bacterial cell growth. J Bacteriol 189, 8575-8583.
170. Deana, A., Celesnik, H., and Belasco, J. G. (2008). The bacterial enzyme RppH triggers messenger RNA degradation by 5′ pyrophosphate removal. Nature 451, 355-358.
171. Danchin, A. (1993). Phylogeny of adenylyl cyclases. Adv Second Messenger Phosphoprotein Res 27, 109-162.
172. Sismeiro, O., Trotot, P., Biville, F., Vivares, C., and Danchin, A. (1998). Aeromonas hydrophila adenylyl cyclase 2: a new class of adenylyl cyclases with thermophilic properties and sequence similarities to proteins from hyperthermophilic archaebacteria. J Bacteriol 180, 3339-3344.
173. Anantharaman, V., and Aravind, L. (2006). The NYN domains: novel predicted RNAses with a PIN domain-like fold. RNA Biol 3, 18-27.
174. Milne, L., Perrin, D. M., and Sigman, D. S. (2001). Oligoribonucleotide-based gene-specific transcription inhibitors that target the open complex. Methods 23, 160-168.
175. Datta, A. K., and Niyogi, K. (1975). A novel oligoribonuclease of Escherichia coli. II. Mechanism of action. J Biol Chem 250, 7313-7319.
176. Sekowska, A., Kung, H. F., and Danchin, A. (2000). Sulfur metabolism in Escherichia coli and related bacteria: facts and fiction. J Mol Microbiol Biotechnol 2, 145-177.
177. Mechold, U., Ogryzko, V., Ngo, S., and Danchin, A. (2006). Oligoribonuclease is a common downstream target of lithium-induced pAp accumulation in Escherichia coli and human cells. Nucleic Acids Res 34, 2364-2373.
178. Zhang, X. X., Lilley, A. K., Bailey, M. J., and Rainey, P. B. (2004). Functional and phylogenetic analysis of a plant-inducible oligoribonuclease (orn) gene from an indigenous Pseudomonas plasmid. Microbiology 150, 2889-2898.
179. Mechold, U., Fang, G., Ngo, S., Ogryzko, V., and Danchin, A. (2007). YtqI from Bacillus subtilis has both oligoribonuclease and pAp-phosphatase activity. Nucleic Acids Res 35, 4552-4561.
180. Oussenko, I. A., Sanchez, R., and Bechhofer, D. H. (2002). Bacillus subtilis YhaM, a member of a new family of 3′-to-5′ exonucleases in gram-positive bacteria. J Bacteriol 184, 6250-6259.
181. Romling, U., and Amikam, D. (2006). Cyclic di-GMP as a second messenger. Curr Opin Microbiol 9, 218-228.
182. Christen, M., Christen, B., Folcher, M., Schauerte, A., and Jenal, U. (2005). Identification and characterization of a cyclic di-GMP-specific phosphodiesterase and its allosteric control by GTP. J Biol Chem 280, 30829-30837.
183. Gupta, R. S. (2000). The natural evolutionary relationships among prokaryotes. Crit. Rev Microbiol 26, 111-131.
184. Yamagata, A., Kakuta, Y., Masui, R., and Fukuyama, K. (2002). The crystal structure of exonuclease RecJ bound to Mn2+ ion suggests how its characteristic motifs are involved in exonuclease activity. Proc Natl Acad Sci USA 99, 5908-5912.
185. Padmanabhan, S., Zhou, K., Chu, C. Y., Lim, R. W., and Lim, L. W. (2001). Overexpression, biophysical characterization, and crystallization of ribonuclease I from Escherichia coli, a broad-specificity enzyme in the RNase T2 family. Arch Biochem Biophys 390, 42-50.
186. Brezellec, P., Hoebeke, M., Hiet, M. S., Pasek, S., and Ferat, J. L. (2006). DomainSieve: a protein domain-based screen that led to the identification of dam-associated genes with potential link to DNA maintenance. Bioinformatics 22, 1935-1941.
187. Oussenko, I. A., Sanchez, R., and Bechhofer, D. H. (2004). Bacillus subtilis YhcR, a high-molecular-weight, nonspecific endonuclease with a unique domain structure. J Bacteriol 186, 5376-5383.
188. Condon, C. (2006). Shutdown decay of mRNA. Mol Microbiol 61, 573-583.
189. Masuda, Y., Miyakawa, K., Nishimura, Y., and Ohtsubo, E. (1993). chpA and chpB, Escherichia coli chromosomal homologs of the pem locus responsible for stable maintenance of plasmid R100. J Bacteriol 175, 6850-6856.
190. Munoz-Gomez, A. J., Santos-Sierra, S., Berzal-Herranz, A., Lemonnier, M., and Diaz-Orejas, R. (2004). Insights into the specificity of RNA cleavage by the Escherichia coli MazF toxin. FEBS Lett 567, 316-320.
191. Tsuchimoto, S., and Ohtsubo, E. (1993). Autoregulation by cooperative binding of the PemI and PemK proteins to the promoter region of the pem operon. Mol Gen Genet. 237, 81-88.
192. Pellegrini, O., Mathy, N., Gogos, A., Shapiro, L., and Condon, C. (2005). The Bacillus subtilis ydcDE operon encodes an endoribonuclease of the MazF/PemK family and its inhibitor. Mol Microbiol 56, 1139-1148.
193. Pandey, D. P., and Gerdes, K. (2005). Toxin-antitoxin loci are highly abundant in free-living but lost from host-associated prokaryotes. Nucleic Acids Res 33, 966-976.
194. Otsuka, Y., and Yonesaki, T. (2005). A novel endoribonuclease, RNase LS, in Escherichia coli. Genetics 169, 13-20.
195. Hecker, M., and Volker, U. (2004). Towards a comprehensive understanding of Bacillus subtilis cell physiology by physiological proteomics. Proteomics 4, 3727-3750.
196. Danchin, A. (2007). Archives or palimpsests? Bacterial genomes unveil a scenario for the origin of fife. Biological Theory 2, 52-61.

Obviously, numerous modifications and variations of the present invention are possible in light of the above teachings. It is therefore to be understood that within the scope of the appended claims, the invention may be practiced otherwise than as specifically described herein.

1. A method of assessing the ageing process of a cell lineage, the method comprising
assessing flux and/or rigidity of at least one member selected from the group consisting of metabolic pathway, conformation of a protein (proline isomer), absence of isoaspartates in a protein, degradation of mRNA, tRNA, and/or rRNA, and activities of one or more of RNase, RTP, polyphosphorylases, Ribonuclease PH, poly(A) polymerase, polynucleotide phosphorylase, ATP-dependent RNA helicases, polyphosphate synthase, polyphosphate kinase, polyphosphate enolase, ribonuclease G, ribonuclease E, Ribonuclease M5, Metallo-β-lactamses, RNase Z, RnjA/B, ribonuclease III, ribonuclease MrnC, ribonuclease H1, ribonuclease HII, ribonuclease HIII, ribonuclease II, ribonuclease R, ribonuclease D, ribonuclease T, mRNA 5′-pyrophosphatase, nanoRNases, and combinations of these in a cell lineage at a first time point,
assessing flux and/or rigidity of at least one metabolic pathway, conformation of a protein (proline isomer), absence of isoaspartates in a protein, RNA degradation of mRNA, tRNA, and/or rRNA, and/or one or more of the activities in a cell lineage at a second time point and/or a time point subsequent to the first time point, and
correlating a change of those assessed items in a cell lineage at a second time point and/or a time point subsequent to the first time point relative to the first time point, a change being indicative of cellular ageing.

2. The method of claim 1, wherein the cell lineage is prokaryotic or eukaryotic.

3. The method of claim 1, wherein the cell lineage is prokaryotic.

4. The method of claim 1, wherein the cell lineage is eukaryotic.

5. The method of claim 4, wherein the eukaryotic cell lineage is mammalian.

6. The method of claim 4, wherein the eukaryotic cell lineage is human.

7. A method of identifying an agent that effects the biological ageing process of a cell lineage; the method comprising
assessing flux and/or rigidity of at least one member selected from the group consisting of metabolic pathway, conformation of a protein (proline isomer), absence of isoaspartates in a protein, degradation of mRNA, tRNA, and/or rRNA, and activities of one or more of RNase, RTP, polyphosphorylases, Ribonuclease PH, poly(A) polymerase, polynucleotide phosphorylase, ATP-dependent RNA helicases, polyphosphate synthase, polyphosphate kinase, polyphosphate enolase, ribonuclease G, ribonuclease E, Ribonuclease M5, Metallo-β-lactamses, RNase Z, RnjA/B, ribonuclease III, ribonuclease MrnC, ribonuclease H1, ribonuclease HII, ribonuclease HIII, ribonuclease II, ribonuclease R, ribonuclease D, ribonuclease T, mRNA 5′-pyrophosphatase, nanoRNases, and combinations of these in a cell lineage at a first time point,
contacting the cells of the cell lineage after the first time point with the agent,
assessing flux and/or rigidity of at least one metabolic pathway, conformation of a protein (proline isomer), absence of isoaspartates in a protein, RNA degradation of mRNA, tRNA, and/or rRNA, and/or one or more of the activities in a cell lineage subsequent to said contacting, and
correlating a change of those assessed items in a cell lineage at the subsequent time point relative to the cells in the cell lineage that have not been contacted, a change being indicative that the agent effects cellular ageing.
